(19) United States
(12) Patent Application Publication
(10) Pub. No.: US 2015/0337379 A1
(43) Pub. Date: Nov. 26, 2015

(54) GENETIC POLYMORPHISMS PREDICTIVE OF NUTRITIONAL REQUIREMENTS FOR CHOLINE IN SUBJECTS

(71) Applicant: The University of North Carolina at Chapel Hill, Chapel Hill, NC (US)

(72) Inventor: Steven H. Zeisel, Chapel Hill, NC (US)

(21) Appl. No.: 14/722,836

(22) Filed: May 27, 2015

Related U.S. Application Data
(63) Continuation of application No. 11/992,709, filed on Jul. 19, 2010.
(60) Provisional application No. 60/723,979, filed on Oct. 5, 2005.

(51) Int. Cl. C12Q 1/68; A61K 31/14; A61K 31/519

(52) U.S. Cl. CPC C12Q 1/6883; A61K 31/519; A61K 31/14; C12Q 2600/156; C12Q 2600/16

(57) ABSTRACT

Methods of predicting susceptibility of a subject to develop one or more choline deficiency-associated health effects are provided, comprising determining a genotype of the subject with respect to at least one choline metabolism gene and comparing the genotype of the subject with at least one reference genotype associated with susceptibility to develop the one or more choline deficiency-associated health effects.

### RELATED APPLICATIONS

This application is a continuation of U.S. patent application Ser. No. 11/992,709, filed on Jul. 19, 2010, which is a national stage of PCT International Application No. PCT/US2006/038887, filed on Oct. 5, 2006, the disclosure of each of which is incorporated herein by reference in its entirety, which is based on and claims priority to U.S. Provisional Patent Application Ser. No. 60/723,979, filed Oct. 5, 2005, the disclosure of which is incorporated herein by reference in its entirety.

### GOVERNMENT INTEREST

This invention was made with government support under Grant Nos. DK55865, AG09525, ES012997, RR00046, and ES10126 awarded by the National Institutes of Health. The government has certain rights in the invention.

### TECHNICAL FIELD

The presently disclosed subject matter relates to predicting the susceptibility of a subject to develop one or more choline deficiency-associated health effects based upon determined genotypes of the subject.

### BACKGROUND

Choline is a required nutrient, and the Institute of Medicine and the National Academy of Sciences of the U.S.A. set an adequate intake level for choline of 550 mg/day for men and 425 mg/day for women. Choline or its metabolites are needed for the structural integrity and signaling functions of cell membranes. It is the major source of methyl groups in the diet (one of choline's metabolites, betaine, participates in the methylation of homocysteine to form methionine), and it directly affects cholinergic neurotransmission, transmembrane signaling, and lipid transport/metabolism (Zeisel & Blusztajn (1994)).

One of the clinical consequences of dietary choline deficiency can be the development of fatty liver (hepatosteatosis) (Buchman et al. (1995); Zeisel et al. (1991)), because a lack of phosphatidylcholine limits the export of excess triglyceride from liver (Yao & Vance (1988); Yao & Vance (1989)). Also, choline deficiency induces hepatocyte apoptosis with leakage of alanine aminotransferase from liver into blood (Zeisel et al. (1991); Albright et al. (1996); Albright et al. (2005)). Some subjects, when deprived of choline, develop muscle damage and increased creatine kinase (CK) activity in blood (da Costa et al. (2004)). This effect may be attributable to impaired membrane stability as a consequence of diminished availability of phosphatidylcholine. The rise in blood CK levels can be a surrogate marker for choline depletion status.

Women's dietary requirements for choline are of special interest because deficient maternal dietary intake of choline during pregnancy in humans has been associated with a 4-fold increased risk of having a baby with a neural tube defect (Shaw et al. (2004)). In addition, offering pregnant rodents diets deficient in choline resulted in perturbed brain development in their fetuses (Albright et al. (1999a); Albright et al. (1999b); Jones et al. (1999); Meck & Williams (1999)).

The factors that influence different dietary requirements for choline in animals, including humans, are not completely understood. Variation between individuals in activity levels of, and interactions between, proteins involved with choline metabolism can potentially affect dietary requirements, which in turn can result from genetic variation of genes encoding choline metabolism proteins. Thus, there is an unmet need for characterization of how genetic variation in genes encoding choline metabolism proteins can be predictive of nutritional requirements for choline.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments of the presently disclosed subject matter, a method of predicting susceptibility of a subject to develop one or more choline deficiency-associated health effects is provided. In some embodiments, the method comprises determining a genotype of the subject with respect to at least one choline metabolism gene and comparing the genotype of the subject with at least one reference genotype associated with susceptibility to develop the one or more choline deficiency-associated health effects, wherein the reference genotype is at least one genotype of a choline metabolism gene.

In some embodiments of the presently disclosed subject matter, a method of treating one or more choline deficiency-associated health effects in a subject is provided. In some embodiments the method comprises determining a genotype of the subject with respect to at least one choline metabolism gene; comparing the determined genotype of the subject with at least one reference genotype associated with susceptibility to develop one or more choline deficiency-associated health effects, wherein the reference genotype is at least one genotype of a choline metabolism gene; and administering to the subject an effective amount of a choline supplement composition, based on the determined genotype being associated with susceptibility to develop one or more choline deficiency-associated health effects.

In some embodiments of the presently disclosed subject matter, a method of predicting activity of a choline metabolism polypeptide in a subject is provided. In some embodiments, the method comprises determining a genotype of the subject with respect to at least one choline metabolism gene; and comparing the genotype of the subject with at least one reference genotype associated with activity of a choline metabolism polypeptide, wherein the reference genotype is at least one genotype of a choline metabolism gene.

In some embodiments of the methods disclosed herein, determining the genotype of the subject comprises:

(a) identifying at least one polymorphism of the at least one choline metabolism gene;

(b) identifying at least one haplotype of the at least one choline metabolism gene;

(c) identifying at least one polymorphism unique to at least one haplotype of the at least one choline metabolism gene;

(d) identifying at least one polymorphism exhibiting high linkage disequilibrium to at least one polymorphism unique to the at least one choline metabolism gene;

(e) identifying at least one polymorphism exhibiting high linkage disequilibrium to the at least one choline metabolism gene; or

(f) combinations thereof.

In some embodiments of the methods disclosed herein, the choline metabolism gene is selected from the group including but not limited to phosphatidylethanolamine N-methyltransferase (PEMT), choline dehydrogenase (CHDH), 5,10-methylenetetrahydrofolate dehydrogenase 1 (MTHFD1), betaine:homocysteine methyltransferase (BHMT), 5,10-methylene tetrahydrofolate reductase (MTHFR), reduced folate carrier 1 (RFC1), ATP-binding cassette sub-family B member 4 (ABCB4), solute carrier family 44 member 1 (SLC44A1), choline kinase alpha (CHKA), and choline kinase beta (CHKB) and combinations thereof. Further, in some embodiments, the reference genotype is selected from the group including but not limited to a PEMT genotype, a CHDH genotype, an MTHFD1 genotype, a BHMT genotype, an MTHFR genotype, a RFC1 genotype, an ABCB4 genotype, a SLC44A1 genotype, a CHKA genotype, a CHKB genotype, and combinations thereof.

In some embodiments of the methods disclosed herein, the reference genotype is a PEMT genotype comprising a G-774C (rs12325817) polymorphism. In some embodiments, the determined genotype of the subject with respect to PEMT comprises at least one copy of a PEMT rs12325817 C allele. In some embodiments, the reference genotype is a CHDH genotype comprising a G432T (rs12676) polymorphism. In some embodiments, the determined genotype of the subject with respect to CHDH comprises at least one copy of a CHDH rs12676 T allele. In some embodiments, the reference genotype is a CHDH genotype comprising a A318C (rs9001) polymorphism. In some embodiments, the determined genotype of the subject with respect to CHDH comprises at least one copy of a CHDH rs9001 C allele. In some embodiments, the reference genotype is a MTHFD1 genotype comprising a G1958A (rs2236225) polymorphism. In some embodiments, the determined genotype of the subject with respect to MTHFD1 comprises at least one copy of a MTHFD1 rs2236225 A allele.

In some embodiments of the methods disclosed herein, the one or more choline deficiency-associated health effects are selected from the group including but not limited to transmembrane signaling dysfunction, cholinergic neurotransmission dysfunction, lipid transport dysfunction, lipid metabolism dysfunction, organ dysfunction, liver dysfunction, fatty liver, congenital birth defects, and combinations thereof. In some embodiments, the one or more choline deficiency-associated health effects are associated with an insufficient dietary intake of choline by the subject. In some embodiments, the subject is the subject is a premenopausal female subject. In some embodiments, the subject is a pregnant subject and the one or more choline deficiency-associated health effects comprise one or more congenital birth defects (e.g., neural tube defects) to a fetus carried by the subject. In other embodiments, the subject is receiving substantially all nutritional sustenance parenterally and the one or more choline deficiency-associated health effects comprise liver dysfunction.

Accordingly, it is an object of the presently disclosed subject matter to provide methods for predicting and/or treating one or more choline deficiency-associated health effects in a subject. This and other objects are achieved in whole or in part by the presently disclosed subject matter.

An object of the presently disclosed subject matter having been stated hereinabove, other aspects and objects will become evident as the description proceeds when taken in connection with the accompanying Drawings and Examples as best described herein below.

### BRIEF DESCRIPTION OF THE DRAWINGS

FIG. 1 is a diagram showing three genes involved in choline metabolism for which single nucleotide polymorphisms were identified. PEMT=phosphatidylethanolamine N-methyltransferase, which catalyzes the reaction to make phosphatidylcholine (PtdCho) from phosphatidylethanolamine (PtdEtn) using S-adenosylmethionine (SAM) to donate methyl groups; CHDH=choline dehydrogenase, which along with betaine aldehyde dehydrogenase irreversibly oxidizes choline (Cho) to form betaine (Bet); BHMT=betaine:homocysteine methyltransferase, which donates its methyl group to homocysteine (Hcy) to form methionine (Met); PCho=phosphocholine.

FIG. 2 is a diagram showing three polymorphic genes that are involved in folate-mediated one-carbon transfer. THF, tetrahydrofolate; MTHFR, 5,10-methylene tetrahydrofolate reductase; MTHFD1, cytosolic 5,10-methylene tetrahydrofolate dehydrogenase; and RFC1, reduced folate carrier 1.

FIG. 3 is a bar graph showing an increase in SAH concentrations after a methionine load was lower in MTFD1 1958 GG individuals. Subjects were treated with a low-choline diet as described in Materials and Methods for Examples 1-3. Blood for SAM and SAH measurements was obtained before (fasting) and 4 hours after an oral methionine load (Met-load; 100 mg of L-methionine per kg of body weight) from 26 individuals with MTHFD1 1958 GA/AA genotype and from 15 individuals with MTHFD1 1958 GG genotype. Values are presented as mean+/−standard error. Solid bars indicate means of individuals with the MTHFD1 1958 GA or AA genotypes, and open bars correspond to means from those with the GG genotype. *, P<0.05; **, P<0.01 different from other genotype by one-way ANOVA.

FIG. 4 is a diagram showing the study design for Example 4. Healthy men and women were fed a baseline diet containing a defined choline adequate intake concentration as defined by the U.S. Institute of Medicine for 10 days. They were then switched to a low choline diet (<50 mg choline) until they developed signs of organ dysfunction associated with choline deficiency or for up to 42 days. Subjects who developed signs of organ dysfunction were repleted with graded amounts of choline at 10 day intervals until their symptoms disappeared; those without signs of organ dysfunction were fed the 100% choline diet for at least 3 days before being discharged from the study. Some subjects were given a folic acid supplement (400 μg per day) during the depletion and repletion phases, but this did not affect their susceptibility to choline deficiency.

### BRIEF DESCRIPTION OF THE TABLES

Table 1 shows a list of exemplary single nucleotide polymorphisms from choline metabolism genes that can be connected with choline deficiency-associated health effects.

Table 2 shows an exemplary research diet menu including actual amounts of food provided for a 2500 kilocalorie diet containing varying amounts of choline. *Percentages show the approximate amount of choline based on the AI. **Wheat starch bread given on Depletion Diet and lecithin bread on Repletion Diets.

Table 3 shows effects of genotype on susceptibility to organ dysfunction in humans eating low-calorie diets. Significance was calculated with a 2×3 Fisher's exact test. Application of Bonferroni's correction for multiple testing lowers the threshold for statistical significance to 0.0125.

Table 4 shows effects of folate and sex on effect of MTHFD1 1958 SNP on susceptibility to organ dysfunction in humans eating low-choline diets. The odds ratios were calculated as the odds of showing signs of deficiency for subjects without the MTHFD1 1958A allele divided by the odds of showing signs of deficiency for subjects with the A allele. Two-sided P and 95% CI were calculated with Fisher's exact test. The odds ratio for premenopausal women was calculated by adding a value of 0.5 to each cell for premenopausal women.

Table 5 lists SNPs studied in Example 4. Each SNP is mapped to the genome and assigned a reference SNP (RefSNP) accession ID (rs number). Base pair and sequence changes, also listed, are subject to revision when genes are resequenced. Note (b): PEMT SNP base pair numbers are numbered from transcription start site (Shields et al. (2001)).

Table 6 lists primers for sequencing the PEMT promoter region. Primers were used to sequence the PEMT gene as described in the Materials and Methods of Example 4.

Table 7 lists effects of PEMT promoter SNP rs12325817 (G-744C) on susceptibility to organ dysfunction in humans eating a low choline diet. Subjects were fed a diet low in choline, and some developed signs of organ dysfunction (liver or muscle) that were reversed when choline was added back to their diets. Numbers of subjects are indicated for each genotype. Two-sided P values were calculated with a 2×3 Fisher exact test. For P<0.05, odds ratios (OR) and 95% confidence intervals (CI) were calculated as the odds of showing signs of deficiency for subjects with the C allele divided by the odds of showing signs of deficiency for subjects without the C allele. Note (b): For postmenopausal and premenopausal women (where some cells were 0), the odds ratio and 95% confidence intervals were computed after adding 0.5 to each cell, so these values underestimate the true values.

Table 8 lists effects of choline dehydrogenase (CHDH) genotypes on susceptibility to organ dysfunction in humans eating a low choline diet. Subjects were fed a diet low in choline and some developed signs of organ dysfunction (liver or muscle), which reversed when choline was added back to their diets. Numbers of subjects are indicated for each genotype. Two-sided P values were calculated with a 2×3 Fisher exact test. For P<0.05, odds ratios (OR) and 95% confidence intervals (CI) were calculated as the odds of showing signs of deficiency for subjects with the C allele (T allele for CHDH 432) divided by the odds of showing signs of deficiency for subjects without the C allele (T allele for CHDH 432).

Table 9 lists data showing PEMT rs7946 (G5465A) and BHMT rs3733890 (G742A) genotypes were not associated with changes in susceptibility to organ dysfunction in humans eating a low choline diet. Subjects were fed a diet low in choline and some developed signs of organ dysfunction (liver or muscle) that reversed when choline was added back to their diets. Numbers of subjects are indicated for each genotype. Two-sided P values were calculated with a 2×3 Fisher exact test. PEMT=phosphatidylethanolamine N-methyltransferase; BHMT=betaine:homocysteine methyltransferase.

### DETAILED DESCRIPTION

Factors that influence the dietary requirement for choline in animals include dietary availability of methyl donors (Zeisel & Blusztajn (1994)) other than choline and endogenous de novo biosynthesis of choline moieties (Bremer & Greenberg (1961)), for example. Each of these factors and others are further influenced by individual genetic variability within genes involved in choline metabolism. As such, some subjects deplete quickly when fed a low-choline diet, whereas others do not. The presently disclosed subject matter provides for the determination of genetic polymorphisms in subjects, which can be utilized to predict individual choline needs and therefore susceptibility to adverse health effects resulting from choline deficiency.

Endogenous production of choline during phosphatidylcholine biosynthesis (through the methylation of phosphatidylethanolamine by phosphatidylethanolamine N-methyltransferase) is most active in liver, but has been identified in many other tissues, including the brain and the mammary gland (Vance et al. (1997); Blusztajn et al. (1985); Yang et al. (1988)). This synthesis of choline provides some, but not all of the choline required to sustain normal organ function in humans (Zeisel et al. (1991)).

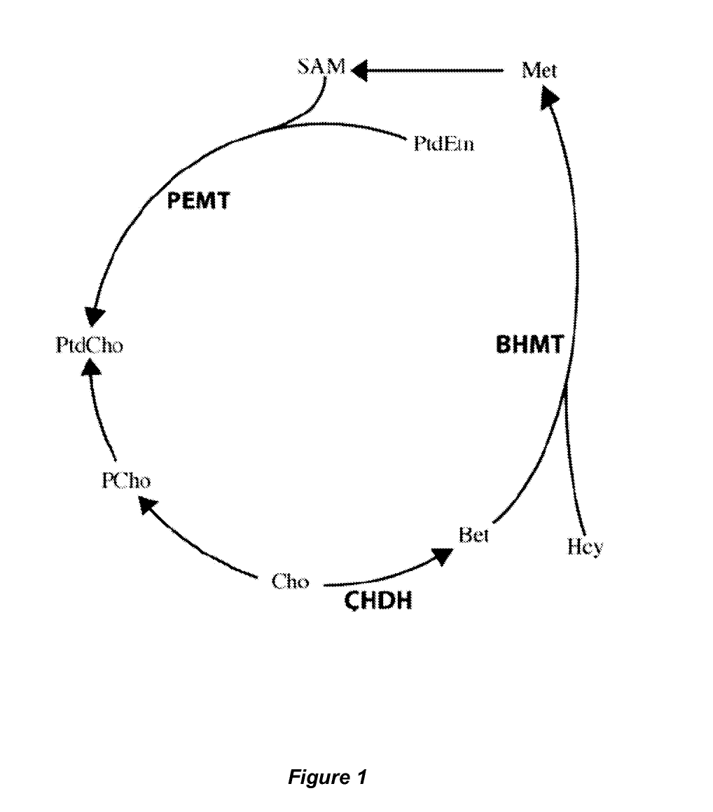

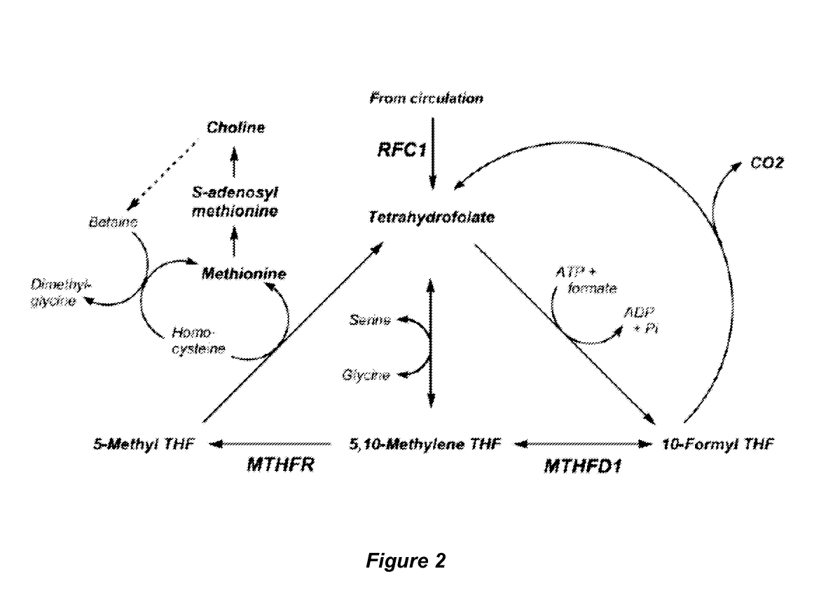

The use of choline as a methyl-group donor also influences the dietary requirement for choline. For example, the methylation of homocysteine to form methionine can be accomplished by using a methyl group derived from one-carbon metabolism or by using a methyl group derived from choline. When choline is used as a methyl group source (see FIGS. 1 and 2), it is first irreversibly oxidized to form betaine by choline dehydrogenase (CHDH) and is no longer available for, for example, synthesis of membrane phosphatidylcholine.

The metabolism of choline, methionine, and methylfolate are closely interrelated and intersect at the formation of methionine from homocysteine (FIG. 2). Betaine:homocysteine methyltransferase (BHMT) catalyzes the remethylation of homocysteine by using the choline metabolite betaine as the methyl donor (Sunden et al. (1997); Millian & Garrow (1998)). In an alternative pathway, 5-methyltetrahydrofolate:homocysteine S-methyltransferase (also known as methionine synthase) regenerates methionine by using a methyl group derived de novo from the one-carbon pool (Bailey & Gregory (1999)). Perturbing the metabolism of one of the methyl donors results in compensatory changes in the other methyl donors because of the intermingling of these metabolic pathways (Kim et al. (1995); Selhub et al. (1991); Varela-Moreiras et al. (1992); Zeisel et al. (1989)).

For example, rats ingesting a low-choline diet showed diminished tissue concentrations of methionine and S-adenosylmethionine (SAM) (Zeisel et al (1989)) and of total folate (Selhub et al. (1991)). Humans deprived of dietary choline have difficulty removing homocysteine after a methionine load and develop elevated plasma homocysteine concentrations (da Costa et al. (2005)). Methotrexate, which is widely used in the treatment of cancer, psoriasis, and rheumatoid arthritis, limits the availability of methyl groups by competitively inhibiting dihydrofolate reductase, a key enzyme in intracellular folate metabolism. Rats treated with methotrexate have diminished pools of all choline metabolites in liver (Pomfret et al. (1990)). Choline supplementation reverses the fatty liver caused by methotrexate administration (Freeman-Narrod et al. (1977); Aarsaether et al. (1988); Svardal et al. (1988)). Genetically modified mice with defective 5,10-methylene tetrahydrofolate reductase (MTHFR) activity become choline-deficient (Schwahn et al. (2003)), a significant observation because many animals, including humans, have genetic polymorphisms that alter the activity of this enzyme (Rozen, R. (1996); Wilcken et al. (1996)).

As noted, genetic variations (e.g., single nucleotide polymorphisms (SNPs)) exist in choline metabolism genes in animals, including humans. However, those genetic variations that have functional effects on choline metabolism, and thereby the nutritional requirements for choline, have not been identified prior to the presently disclosed subject matter. For example, if decreased availability of methyl groups from choline is responsible for organ dysfunction in choline deficiency, then particular SNPs in CHDH or BHMT could possibly alter susceptibility to developing organ dysfunction when fed a low choline diet. Alternatively, if organ damage is due to defective membrane formation, SNPs in PEMT encoding phosphatidylethanolamine N-methyltransferase (PEMT), which catalyzes phosphatidylcholine formation from SAM (FIG. 1), can modify de novo phosphatidylcholine synthesis and SNPs resulting in decreased CHDH activity could decrease the use of choline as a methyl donor and make more substrate available for phosphatidylcholine synthesis from preexisting choline moiety, thereby altering susceptibility to developing organ dysfunction when fed a low choline diet. Thus, SNPs in genes involved in choline metabolism, including folate metabolism, can potentially increase the demands for choline as a methyl-group donor, thereby increasing dietary requirements for this essential nutrient.

The presently disclosed subject matter provides new insights into the molecular pathways involved in the development of choline deficiency-associated health effects and further reveals genotypes, including genetic polymorphisms present in subjects, which can produce a clinical phenotype that is vulnerable to the development of one or more choline deficiency-associated health effects. The genotypes (which can include genetic polymorphisms) identified herein are useful for predicting the susceptibility of a subject to develop one or more choline deficiency-associated health effects, including for example organ dysfunction and congenital birth defects. The disclosed genotypes can also be utilized to predict the expression level and/or activity of peptides encoded by one or more choline metabolism genes.

The presently disclosed subject matter also provides methods for utilizing the knowledge of the genotype (which can include the presence of polymorphisms (see e.g., Table 1) of a particular subject to treat the subject for one or more choline deficiency-associated health effects. The treatment can be administered to the subject either before the onset of symptoms and in anticipation thereof based on a determined genotype of the subject, or after occurrence of symptoms associated with choline deficiency-associated health effects and confirmation of a susceptible genotype in the subject.

Therefore, determining a subject's genotype for choline metabolism genes, including but not limited to phosphatidylethanolamine N-methyltransferase (PEMT), choline dehydrogenase (CHDH), betaine:homocysteine methyltransferase (BHMT), methylenetetrahydrofolate dehydrogenase 1 (MTHFD1), 5,10-methylene tetrahydrofolate reductase (MTHFR), reduced folate carrier 1 (RFC1), ATP-binding cassette sub-family B member 4 (ABCB4), solute carrier family 44 member 1 (SLC44A1), choline kinase alpha (CHKA), choline kinase beta (CHKB), and combinations thereof can be used to predict the susceptibility of the subject to develop choline deficiency-associated health effects, predict the activity of a peptide encoded by one or more choline metabolism genes, and/or to select effective treatments for the subject, as disclosed in detail herein.

### I. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

A “choline metabolism gene” as used herein refers to a polynucleotide expressing a protein that functions, at least in part, in the metabolism of choline. “Choline metabolism” as used herein is intended to encompass all physiological aspects of choline production, function, and degradation, including but not limited to choline synthesis and catabolism, as well as choline use within other metabolic pathways, including for example the physiological utilization of choline in methyl donation reactions (e.g., folate-mediated one-carbon transfer). Choline and folate metabolism are interrelated and therefore, the term “choline metabolism” is intended to include folate metabolism as well. Exemplary non-limiting pathways of choline metabolism are shown in FIGS. 1 and 2 and each of the proteins disclosed in these pathways are specifically intended to be included within the definition of a protein that functions in the metabolism of choline (i.e., a choline metabolism protein). Thus, in some embodiments a choline metabolism gene is a polynucleotide encoding, for example, PEMT, CHDH, MTHFD1, BHMT, MTHFR, RFC1, ABCB4, SLC44A1, CHKA, or CHKB.

“PEMT gene” as used herein refers in some embodiments to a gene encoding a phosphatidylethanolamine N-methyltransferase protein (PEMT) and/or associated regulatory sequences. PEMT transfers methyl groups between molecules and can function to catalyze a reaction to produce phosphatidylcholine (PtdCho) from phosphatidylethanolamine (PtdEtn) using S-adenosylmethionine (SAM) to donate methyl groups. An exemplary PEMT gene can be a human PEMT gene located within a PEMT locus on chromosome 17 (GENBANK® Accession No. NC_{—}000017) between about nucleotide positions 17,349,830 and 17,435,665.

“CHDH gene” as used herein refers in some embodiments to a gene encoding a choline dehydrogenase protein (CHDH) and/or associated regulatory sequences. CHDH can function to irreversibly oxidize choline to form betaine. An exemplary CHDH gene can be a human CHDH gene located within a CHDH locus on chromosome 17 (GENBANK® Accession No. NC_{—}000003) between about nucleotide positions 53,826,844 and 53,833,075.

“MTHFD1 gene” as used herein refers in some embodiments to a gene encoding a cytosolic 5,10-methylene tetrahydrofolate dehydrogenase (MTHFD1) protein and/or associated regulatory sequences. MTHFD1 catalyzes the transfer of hydrogens from donor to acceptor molecules. MTHFD1 can catalyze the conversion of 5,10-methylene tetrahydrofolate to 10-formyl tetrahydrofolate, and vice versa. An exemplary MTHFD1 gene can be a human MTHFD1 gene located within a MTHFD1 locus on chromosome 14 (GENBANK® Accession No. NC_{—}000014) between about nucleotide positions 63,924,899 and 63,994,774.

“BHMT” gene as used herein refers in some embodiments to a gene encoding a betaine:homocysteine methyltransferase (BHMT) protein and/or associated regulatory sequences. BHMT catalyzes the transfer of a methyl group to homocysteine from betaine to form methionine. An exemplary BHMT gene can be a human BHMT gene located within a BHMT locus on chromosome 5 (GENBANK® Accession No. NC_{—}000005) between about nucleotide positions 78,443,465 and 78,462,695.

“MTHFR” gene as used herein refers in some embodiments to a gene encoding a 5,10-methylene tetrahydrofolate reductase (MTHFR) protein and/or associated regulatory sequences. MTHFR can catalyze the conversion of 5,10-methylene tetrahydrofolate to 5-methyl tetrahydrofolate. An exemplary MTHFR gene is a human MTHFR gene located within a MTHFR locus on chromosome 1 (GENBANK® Accession No. NC_{—}000001) between about nucleotide positions 11,773,324 and 11,785,760.

“RFC1” gene as used herein refers in some embodiments to a gene encoding a reduced folate carrier 1 (RFC1) protein and/or associated regulatory sequences. RFC1 can function, for example, as a folate transporter protein. An exemplary RFC1 gene is a human RFC1 gene located within a RFC1 locus on chromosome 4 (GENBANK® Accession No. NC_{—}000004).

“ABCB4” gene as used herein refers in some embodiments to a gene encoding a ATP-binding cassette, sub-family B, member 4 (ABCB4) protein and/or associated regulatory sequences. ABCB4 is a transmembrane protein that can bind ATP and use the energy to drive the transport of various molecules across all cell membranes. An exemplary ABCB4 gene is a human ABCB4 gene located within a ABCB4 locus on chromosome 7 (GENBANK® Accession No. NC_{—}000007) between about nucleotide positions 86,869,348 and 86,942,717.

“SLC44A1” gene as used herein refers in some embodiments to a gene encoding a solute carrier family 44, member 1 (SLC44A1) protein and/or associated regulatory sequences. SLC44A1 can transport choline molecules. An exemplary SLC44A1 gene is a human SLC44A1 gene located within a SLC44A1 locus on chromosome 9 (GENBANK® Accession No. NC_{—}000009).

“CHKA” gene as used herein refers in some embodiments to a gene encoding a choline kinase alpha (CHKA) protein and/or associated regulatory sequences. CHKA can phosphorylate choline molecules. An exemplary CHKA gene is a human CHKA gene located within a CHKA locus on chromosome 11 (GENBANK® Accession No. NC_{—}000011) between about nucleotide positions 67,567,632 and 67,645,220.

“CHKB” gene as used herein refers in some embodiments to a gene encoding a choline kinase beta (CHKB) protein and/or associated regulatory sequences. CHKB can phosphorylate choline molecules. An exemplary CHKB gene is a human CHKB gene located within a CHKB locus on chromosome 22 (GENBANK® Accession No. NC_{—}000022) between about nucleotide positions 49,364,476 and 49,368,076.

As used herein, the term “expression” generally refers to the cellular processes by which an RNA is produced by RNA polymerase (RNA expression) or a polypeptide is produced from RNA (protein expression).

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

As used herein, the term “DNA segment” means a DNA molecule that has been isolated free of total genomic DNA of a particular species. Included within the term “DNA segment” are DNA segments and smaller fragments of such segments, and also recombinant vectors, including, for example, plasmids, cosmids, phages, viruses, and the like.

As used herein, the term “genotype” means the genetic makeup of an organism. Expression of a genotype can give rise to an organism's phenotype, i.e. an organism's physical traits. The term “phenotype” refers to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof. The phenotype can be related to choline metabolism and/or choline deficiency-associated health effects. As such, a subject's genotype when compared to a reference genotype or the genotype of one or more other subjects can provide valuable information related to current or predictive phenotypes.

“Determining the genotype” of a subject, as used herein, can refer to determining at least a portion of the genetic makeup of an organism and particularly can refer to determining a genetic variability in the subject that can be used as an indicator or predictor of phenotype. The genotype determined can be the entire genome of a subject, but far less sequence is usually required. The genotype determined can be as minimal as the determination of a single base pair, as in determining one or more polymorphisms in the subject (see e.g., Table 1). Further, determining a genotype can comprise determining one or more haplotypes. Still further, determining a genotype of a subject can comprise determining one or more polymorphisms exhibiting high linkage disequilibrium to at least one polymorphism or haplotype having genotypic value.

As used herein, the term “polymorphism” refers to the occurrence of two or more genetically determined alternative variant sequences (i.e., alleles) in a population. A polymorphic marker is the locus at which divergence occurs. Preferred markers have at least two alleles, each occurring at a frequency of greater than 1%. A polymorphic locus may be as small as one base pair (e.g., a single nucleotide polymorphism (SNP)). Exemplary SNPs are disclosed herein and can be referenced by accession number (e.g., “rs number”) and/or SEQ ID NO. Both rs numbers (searchable through NCBI's Entrez SNP website) and SEQ ID NOs comprise the SNP as well as proximate contiguous nucleotides provided to place the SNP in context within the gene. Thus, rs numbers and/or SEQ ID NOs referenced herein are intended to indicate the presence of the SNP and not to require the presence of all or part of the contiguous nucleotide sequence disclosed therein. Further, reference to a particular polymorphism is intended to also encompass the complementary nucleotide(s) on the complementary nucleotide strand (e.g., coding and non-coding polynucleotides).

As used herein, “haplotype” means the collective characteristic or characteristics of a number of closely linked loci with a particular gene or group of genes, which can be inherited as a unit. For example, in some embodiments, a haplotype can comprise a group of closely related polymorphisms (e.g., single nucleotide polymorphisms (SNPs)). In some embodiments, the determined genotype of a subject can be particular haplotypes for one or more of PEMT, CHDH, BHMT, MTHFD1, MTHFR, RFC1, ABCB4, SLC44A1, CHKA, and CHKB.

As used herein, “linkage disequilibrium” (LD) means a derived statistical measure of the strength of the association or co-occurrence of two independent genetic markers. Various statistical methods can be used to summarize LD between two markers but in practice only two, termed D′ and r2, are widely used, as is generally known in the art.

In some embodiments, determining the genotype of a subject can comprise identifying at least one polymorphism (e.g., an SNP) of at least one gene, such as for example PEMT, CHDH, BHMT, MTHFD1, MTHFR, RFC1, ABCB4, SLC44A1, CHKA, CHKB, and combinations thereof. In some embodiments, determining the genotype of a subject can comprise identifying at least one haplotype of a gene, such as for example PEMT, CHDH, BHMT, MTHFD1, MTHFR, RFC1, ABCB4, SLC44A1, CHKA, CHKB, and combinations thereof. In some embodiments, determining the genotype of a subject can comprise identifying at least one polymorphism unique to at least one haplotype of a gene, such as for example PEMT, CHDH, BHMT, MTHFD1, MTHFR, RFC1, ABCB4, SLC44A1, CHKA, CHKB, and combinations thereof. In some embodiments, determining the genotype of a subject can comprise identifying at least one polymorphism exhibiting high linkage disequilibrium to at least one polymorphism unique to at least one haplotype, such as for example PEMT haplotype, CHDH haplotype, BHMT haplotype, MTHFD1 haplotype, MTHFR haplotype, RFC1 haplotype, ABCB4 haplotype, SLC44A1 haplotype, CHKA haplotype, CHKB haplotype, or combinations thereof. In some embodiments, determining the genotype of a subject can comprise identifying at least one polymorphism exhibiting high linkage disequilibrium to at least one haplotype, such as for example PEMT haplotype, CHDH haplotype, BHMT haplotype, MTHFD1 haplotype, MTHFR haplotype, RFC1 haplotype ABCB4 haplotype, SLC44A1 haplotype, CHKA haplotype, CHKB haplotype, or combinations thereof. Table 1 provides an exemplary non-limiting list of SNPs that can be correlated with choline deficiency-associated health effects.

As used herein, the term “modulate” means an increase, decrease, or other alteration of any, or all, chemical and biological activities or properties of a wild-type or mutant polypeptide, such as for example PEMT, CHDH, BHMT, MTHFD1, MTHFR, RFC1, ABCB4, SLC44A1, CHKA, CHKB or combinations thereof. A peptide activity can be modulated at either the level of expression, e.g., modulation of gene expression (for example, due to polymorphisms within the regulatory sequences of a gene), or at the level of protein activity (e.g., polymorphism resulting in amino acid changes affecting protein activity). The term “modulation” as used herein refers to both upregulation (i.e., activation or stimulation) and downregulation (i.e. inhibition or suppression) of an expression level and/or activity level.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

“Choline deficiency-associated health effects” as used herein refers to clinical conditions and symptoms directly or indirectly resulting from insufficient amounts of choline within the particular subject. Amounts of choline required by individual subjects vary depending on multiple factors, including genetic variation between individuals of choline metabolism genes, as disclosed herein. Therefore, the amount of choline required by a particular subject to prevent or treat choline deficiency-associated health effects can vary significantly. Determination of a subject's genotype with regard to choline metabolism gene(s) can help predict susceptibility of a subject to choline deficiency-associated health effects. Exemplary choline deficiency-associated health effects include, but are not limited to transmembrane signaling dysfunction, cholinergic neurotransmission dysfunction, lipid transport dysfunction, lipid metabolism dysfunction, organ dysfunction, liver dysfunction, fatty liver, congenital birth defects, and combinations thereof. Congenital birth defects can include, but are not limited to, neural tube defects (e.g., spina bifida).

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. A p-value in some embodiments less than or equal to 0.05, in some embodiments less than 0.01, in some embodiments less than 0.005, and in some embodiments less than 0.001, are regarded as significant.

TABLE 1

| GENE/SNP | | SEQ |
|---|---|---|
| ID No | Proximate Sequence and [polymorphism] | ID NO |
| PEMT | | |
| rs12325817 | cagcctggacaacatggtgacactc[G/C]gtctctactaaaaatacaaaaatag | 1 |
| rs2278952 | CCTGCAGCTCAGCAGACCTCCTGGC[C/T]GTGGTGGGTAGCTCCTTTCCTTTAG | 2 |
| rs7946 | ACTCACTCTTCGTATAGGAGAGCCA[C/T]TATGTAGGTGAGGGCCACCAGCACC | 3 |
| rs8068641 | CGATCCCCTGCATGTGGGGCTCACC[A/G]AGCAGTGGGATACTCCCGTCTGGAC | 4 |
| rs7224725 | CTATCTCTGGCCATACTCTCAGCCA[C/T]CCTAAAAGTAAAGCCTTGTTGTTAG | 5 |
| rs956967 | CAGTTTCCTTGGGTTTCAAGACCCA[A/G]AAGCACTCACTTTTATCCTTGTCCC | 6 |
| rs9944423 | AGATTAAGCAACTAACACTGGGGGA[A/G]CCCAGCAGCAGCTGACATCCACCTG | 7 |
| rs3760188 | TGGGGTGAAGCTCACAACCAGCAGA[A/G]GAGTGAGAAGAGGCAGCCCAGCGTG | 8 |
| rs7215880 | AGGCCCTGCCACCGAGCTGTTCGTT[A/G]CCTCGGCTCCCGGGTTCCCAGATCT | 9 |
| rs7217778 | GGGAGGTGCCAGATGTGCCAAGTGT[G/T]AGGGCAGGGCAAACAAGGCAAGACG | 10 |
| rs7215833 | CAGCTAGGTGATAATTACTAATCTC[C/T]GCTACTGTGTATGGAAGACATCTTG | 11 |
| rs4646341 | CCAGAGAGTTCTCTGAAGGAGCTAA[C/T]ACCAGTTAGTGTTTTGAAGAGTAGC | 12 |
| CHDH | | |
| rs9001 | AGCGCAGGCTCTGAGAGCCGGGACG[A/C]GTACAGCTATGTGGTGGTGGGCGCG | 13 |
| rs12676 | CTGGAGGCCGGGCCCAAGGACGTGC[G/T]CGCGGGGAGCAAGCGGCTCTCGTGG | 14 |
| MTHFD1 | | |
| rs2236225 | TGGGCCAACAAGCTTGAGTGCGATC[C/T]GGTCTGCAATGATGGAGGAATTGCC | 15 |
| ABCB4 | | |
| rs1202283 | GCCGCGTATTGAGTTCAGTGGTGTC[A/G]TTGATGTCAAACCATCCTATTTCCT | 16 |
| rs8187797 | AGAAATTTGACACCCTGGTTGGAGA[C/G]AGAGGGGCCCAGCTGAGTGGTGGGC | 17 |
| rs8187801 | GGCGAGATCCTCACCAGAAGACTGC[A/G]GTCAATGGCTTTTAAAGCAATGCTA | 18 |
| rs8187799 | ACTAAATGATGAAAAGGCTGCCACT[A/G]GAATGGCCCCAAATGGCTGGAAATC | 19 |
| rs8187788 | CATAGCTCACGGATCAGGTCTCCCC[C/G]TCATGATGATAGTATTTGGAGAGAT | 20 |
| rs8187792 | TCACTGTTTCTTTTCTGTCCAGATA[C/G]TCTCGGCATTTAGTGACAAAGAACT | 21 |
| rs8187811 | AGGAGGTCAAAAACAGAGGATTGCT[A/G]TTGCCCGAGCCCTCATCAGACAACC | 22 |
| rs31655 | AGCCACTGGACATTGAGTTTCTTTG[C/T]TTCTTGACCATCGAGAAGCTGAAAA | 23 |
| MTHFR | | |
| rs1801133 | TTGAAGGAGAAGGTGTCTGCGGGAG[C/T]CGATTTCATCATCACGCAGCTTTTC | 24 |
| rs1801131 | TGGGGGGAGGAGCTGACCAGTGAAG[A/C]AAGTGTCTTTGAAGTCTTTGTTCTT | 25 |
| BHMT | | |
| rs3733890 | ATGAAGGAGGGCTTGGAGGCTGCCC[A/G]ACTGAAAGCTCACCTGATGAGCCAG | 26 |
| CHKA | | |
| rs17857113 | CCCAAAGTAGCCTACCAACTCACCA[C/T]CTGCAAAATCGCTCCATACAGCCGC | 27 |
| rs17853642 | AATCTTGGCTTGTACAATGGACCAC[A/T]GTCCCCAGAGGAAATGAGATGCAAG | 28 |
| rs17853641 | ATTTCTGCAGAAATATCTGGCAAAC[C/T]TAATTCTTCAGTATCTAATCGCCGG | 29 |

### II. METHODS OF PREDICTING SUSCEPTIBILITY TO DEVELOP CHOLINE DEFICIENCY-ASSOCIATED HEALTH EFFECTS

The presently disclosed subject matter provides for determining a genotype of a subject with respect to particular genes having a role in choline metabolism. Determining the genotype of the subject with regard to choline metabolism genes can elucidate susceptibility to develop choline deficiency-associated health effects in the subject. The present subject matter discloses that the PEMT, CHDH, BHMT, MTHFD1, MTHFR, RFC1, ABCB4, SLC44A1, CHKA, and CHKB genes encode for proteins that can each, and in combination with one another, play an important role in choline metabolism and individual sensitivity to choline deficiency. Thus, genotyping one or more of these genes can provide valuable information related to choline deficiency sensitivity useful for predicting susceptibility to develop choline deficiency-associated health effects and even insights into effective therapies to treat choline deficiency-associated health effects and related conditions.

On the basis of the data disclosed herein and the related discussion, the presently disclosed subject matter provides methods of predicting susceptibility of a subject, i.e. the predisposition of or risk of the subject, to develop choline deficiency-associated health effects. In some embodiments, the method comprises determining a genotype of the subject with respect to at least one choline metabolism gene, such as for example PEMT, CHDH, BHMT, MTHFD1, MTHFR, RFC1, ABCB4, SLC44A1, CHKA, CHKB, and combinations thereof; and comparing the genotype of the subject with at least one reference genotype associated with susceptibility to develop one or more choline deficiency-associated health effects, whereby susceptibility of the subject to develop the choline deficiency-associated health effects is predicted. In some embodiments, the reference genotype is selected from the group including but not limited to a PEMT genotype, a CHDH genotype, an MTHFD1 genotype, a BHMT genotype, an MTHFD1 genotype, an MTHFR genotype, a RFC1 genotype, an ABCB4 genotype, a SLC44A1 genotype, a CHKA genotype, a CHKB genotype and combinations thereof.

“Reference genotype” as used herein refers to a previously determined pattern of genetic variation associated with a particular phenotype, such as for example choline deficiency-associated health effects. The reference genotype can be as minimal as the determination of a single base pair, as in determining one or more polymorphisms in the subject. Further, the reference genotype can comprise one or more haplotypes. Still further, the reference genotype can comprise one or more polymorphisms exhibiting high linkage disequilibrium to at least one polymorphism or haplotype. In some particular embodiments, the reference genotype comprises one or more polymorphisms (e.g., SNPs) and/or haplotypes of PEMT, CHDH, BHMT, MTHFD1, MTHFR, RFC1, ABCB4, SLC44A1, CHKA, CHKB, or combinations thereof determined to be associated with choline deficiency-associated health effects. In some embodiments, the haplotypes represent a particular collection of specific single nucleotide polymorphisms.

In particular embodiments, the reference genotype is a human PEMT genotype comprising a G-774C (rs12325817; SEQ ID NO: 1) polymorphism, a human CHDH genotype comprising a G432T (rs12676; SEQ ID NO:14) polymorphism, a human CHDH genotype comprising a A318C (rs9001; SEQ ID NO:13) polymorphism, a human MTHFD1 genotype comprising a G1958A (rs2236225; SEQ ID NO:15 (note “GI 958A” throughout the present disclosure references the polymorphism on the minus strand, whereas the rs number and SEQ ID NO:15 disclose the complementary strand and reference the polymorphism as C/T)) polymorphism, or a combination thereof. Further, in some embodiments, the determined genotype of the subject with respect to PEMT comprises at least one copy of a PEMT rs12325817 C allele (i.e., a C is present at nucleotide −774 (non-coding region) of at least one copy of the PEMT gene) and the subject is predicted to be susceptible to develop one or more choline deficiency-associated health effects. In some embodiments, the determined genotype of the subject with respect to CHDH comprises at least one copy of a CHDH rs12676 T allele (i.e., a T is present at nucleotide +432 (coding region) of at least one copy of the CHDH gene and the subject is predicted to be susceptible to develop one or more choline deficiency-associated health effects. In some embodiments, the determined genotype of the subject with respect to CHDH comprises at least one copy of a CHDH rs9001 C allele (i.e., a C is present at nucleotide +318 (coding region) of at least one copy of the CHDH gene and the subject is predicted to be resistant to develop the one or more choline deficiency-associated health effects. By “resistant” is meant that the subject is less likely than the average over a population to develop choline deficiency-associated health effects when consuming a diet low in choline over time. In some embodiments, the determined genotype of the subject with respect to MTHFD1 comprises at least one copy of a MTHFD1 rs2236225 A allele (i.e., a A is present at nucleotide +1958 (coding region) of at least one copy of the MTHFD1 gene and the subject is predicted to be susceptible to develop the one or more choline deficiency-associated health effects.

In some embodiments of the methods of predicting susceptibility of a subject to develop one or more choline deficiency-associated health effects disclosed herein, determining the genotype of the subject comprises one or more of:
(i) identifying at least one polymorphism of the at least one choline metabolism gene;
(ii) identifying at least one haplotype of the at least one choline metabolism gene;
(iii) identifying at least one polymorphism unique to at least one haplotype of the at least one choline metabolism gene;
(iv) identifying at least one polymorphism exhibiting high linkage disequilibrium to at least one polymorphism unique to the at least one choline metabolism gene; and
(v) identifying at least one polymorphism exhibiting high linkage disequilibrium to the at least one choline metabolism gene.

The determined genotype of the subject is then compared to one or more reference genotypes associated with susceptibility to develop one or more choline deficiency-associated health effects and if the determined genotype matches the reference genotype, the subject is predicted to be susceptible to a particular degree (as compared to a population norm) to develop one or more choline deficiency-associated health effects.

As indicated above, the determined genotype need not necessarily be determined based on a need to compare the determined genotype to the reference genotype in particular, but rather can be for example one or more polymorphisms exhibiting high linkage disequilibrium to a PEMT, CHDH, BHMT, MTHFD1, MTHFR, RFC1, ABCB4, SLC44A1, CHKA, or CHKB polymorphism or haplotype, or combinations thereof, which can be equally predictive of susceptibility to develop one or more choline deficiency-associated health effects. For example, there are presently more than 98 known SNPs for PEMT (Saito et al. (2001)). Selecting one or more of these SNPs, it is then determined, by art recognized techniques, if one or more of the known SNPs of PEMT exhibit high linkage disequilibrium to one or more of the SNPs used to determine the reference genotypes of PEMT predictive of susceptibility to develop one or more choline deficiency-associated health effects. Thus, after a review of the guidance provided herein, one of ordinary skill would appreciate that any one or more polymorphisms exhibiting high linkage disequilibrium to a polymorphism or haplotype of the determined genotype with regard to PEMT could likewise be effective as a substitute of or additional component for the determined genotype and/or reference genotype. Likewise, polymorphisms exhibiting high linkage disequilibrium to PEMT, CHDH, BHMT, MTHFD1, MTHFR, RFC1, ABCB4, SLC44A1, CHKA, and/or CHKB (i.e. haplotypes and/or polymorphisms) could be used to supplement or replace components of the determined genotype and/or reference genotype.

As disclosed herein, it has been determined that certain polymorphisms correlate particularly well as predictors of susceptibility to develop choline deficiency-associated health effects in female subjects and these correlations can be utilized to determine appropriate treatment options. Thus, in some embodiments, the subject is a female. Further, in some embodiments, the subject is a premenopausal female and the determined genotype of the subject comprises at least one copy of a PEMT rs12325817 C allele, at least one copy of a MTHFD1 rs2236225 A allele, or combinations thereof and the subject is therefore predicted to be susceptible to develop one or more choline deficiency-associated health effects. Further, in some embodiments, the premenopausal female subject predicted to be susceptible to develop one or more choline deficiency-associated health effects is predicted to be susceptible to develop one or more congenital birth defects (e.g., neural tube defects) in a fetus carried by the subject as a result of choline deficiency in the subject.

### III. METHODS OF PREDICTING BIOLOGICAL ACTIVITY OF CHOLINE METABOLISM POLYPEPTIDES

As disclosed herein, polymorphic differences in choline metabolism genes can be predictive of susceptibility to choline deficiency-associated health effects by the subject carrying the polymorphism. In some instances, the polymorphic difference can result in modulation in expression levels of a polypeptide or modulation in biological activity of the expressed polypeptide as compared to a comparable polypeptide expressed from a gene without the polymorphism. Thus, the polymorphic differences disclosed herein can be predictive of the biological activity (including expression levels) of the expressed polypeptides.

As such, the presently disclosed subject matter provides methods of predicting biological activity of a choline metabolism polypeptide in a subject. In some embodiments, the methods comprise determining a genotype of the subject with respect to at least one choline metabolism gene, such as but not limited to PEMT, CHDH, BHMT, MTHFD1, MTHFR, RFC1, ABCB4, SLC44A1, CHKA, CHKB or combinations thereof and comparing the genotype of the subject with at least one reference genotype associated with variability in biological activity of a choline metabolism gene, whereby biological activity of the proteins in the subject is predicted. Variability in biological activity can be either an increase or a decrease in activity (e.g., enzyme activity) of the polypeptide comprising the predictive polymorphism as compared to a similar polypeptide comprising the “normal” allele.

In some embodiments of the methods, determining the genotype of the subject comprises one or more of:
(i) identifying at least one polymorphism of the at least one choline metabolism gene;
(ii) identifying at least one haplotype of the at least one choline metabolism gene;
(iii) identifying at least one polymorphism unique to at least one haplotype of the at least one choline metabolism gene;
(iv) identifying at least one polymorphism exhibiting high linkage disequilibrium to at least one polymorphism unique to the at least one choline metabolism gene; and
(v) identifying at least one polymorphism exhibiting high linkage disequilibrium to the at least one choline metabolism gene.

In some embodiments the reference genotype is selected from the group including but not limited to a PEMT genotype, a CHDH genotype, an MTHFD1 genotype, a BHMT genotype, an MTHFD1 genotype, an MTHFR genotype, a RFC1 genotype, an ABCB4 genotype, a SLC44A1 genotype, a CHKA genotype, and a CHKB genotype and combinations thereof. In particular embodiments, the reference genotype is a human PEMT genotype comprising a G-774C (rs12325817; SEQ ID NO: 1) polymorphism, a human CHDH genotype comprising a G432T (rs12676; SEQ ID NO:14) polymorphism, a human CHDH genotype comprising a A318C (rs9001; SEQ ID NO:13) polymorphism, a human MTHFD1 genotype comprising a G1958A (rs2236225; SEQ ID NO:15), or a combination thereof. Each of these genotypes can be predictive of an activity level of the particular choline metabolism polypeptide.

### IV. METHODS OF TREATMENT

The genotyping methods for predicting susceptibility to develop by a subject choline deficiency-associated health effects disclosed herein are applicable as well to the present methods of treating choline deficiency-associated health effects in a subject. Determining a genotype of a subject with regard to choline metabolism genes can be useful in selecting a particular therapy for use in treating the subject.

As such, in some embodiments of the presently disclosed subject matter methods of treating choline deficiency-associated health effects in a subject are provided. In some embodiments, the methods comprise determining a genotype of the subject with respect to at least one choline metabolism gene, such as but not limited to PEMT, CHDH, BHMT, MTHFD1, MTHFR, RFC1, ABCB4, SLC44A1, CHKA, CHKB, or combinations thereof; comparing the determined genotype of the subject with at least one reference genotype associated with susceptibility to develop one or more choline deficiency-associated health effects, wherein the reference genotype is at least one genotype of a choline metabolism gene; and administering to the subject an effective amount of a choline supplement composition (e.g., a composition comprising choline, folate, or combinations thereof, or any agent that can supplement choline metabolism, as would be apparent to one of ordinary skill in the art upon review of the instant disclosure), based on the determined genotype being associated with susceptibility to develop one or more choline deficiency-associated health effects.

“Treatment” and “treating” as used herein refer to any treatment of one or more choline deficiency-associated health effects and includes: (i) preventing the health effect from occurring in a subject which may be predisposed to the health effect, but has not yet been diagnosed as having it; (ii) inhibiting the health effect, i.e., arresting its further development; or (iii) relieving the health effect, i.e., causing regression of clinical symptoms of the health effect.

In some embodiments of the methods, determining the genotype of the subject comprises one or more of:
(i) identifying at least one polymorphism of the at least one choline metabolism gene;
(ii) identifying at least one haplotype of the at least one choline metabolism gene;
(iii) identifying at least one polymorphism unique to at least one haplotype of the at least one choline metabolism gene;
(iv) identifying at least one polymorphism exhibiting high linkage disequilibrium to at least one polymorphism unique to the at least one choline metabolism gene; and
(v) identifying at least one polymorphism exhibiting high linkage disequilibrium to the at least one choline metabolism gene.

In some embodiments, the reference genotype is selected from the group including but not limited to a PEMT genotype, a CHDH genotype, an MTHFD1 genotype, a BHMT genotype, an MTHFD1 genotype, an MTHFR genotype, a RFC1 genotype, an ABCB4 genotype, a SLC44A1 genotype, a CHKA genotype, a CHKB genotype and combinations thereof. In particular embodiments, the reference genotype is a human PEMTgenotype comprising a G-774C (rs12325817; SEQ ID NO: 1) polymorphism, a human CHDH genotype comprising a G432T (rs12676; SEQ ID NO:14) polymorphism, a human CHDH genotype comprising a A318C (rs9001; SEQ ID NO:13) polymorphism, a human MTHFD1 genotype comprising a G1958A (rs2236225; SEQ ID NO:15), or combinations thereof. Further, in some embodiments, the determined genotype of the subject with respect to PEMT comprises at least one copy of a PEMT rs12325817 C allele (i.e., a C is present at nucleotide −774 (non-coding region) of at least one copy of the PEMT gene) and the subject is administered the composition. In some embodiments, the determined genotype of the subject with respect to CHDH comprises at least one copy of a CHDH rs12676 T allele (i.e., a T is present at nucleotide +432 (coding region) of at least one copy of the CHDH gene and the subject is administered the composition. In some embodiments, the determined genotype of the subject with respect to CHDH comprises at least one copy of a CHDH rs9001 C allele (i.e., a C is present at nucleotide +318 (coding region) of at least one copy of the CHDH gene and the subject is not administered the composition. In some embodiments, the determined genotype of the subject with respect to MTHFD1 comprises at least one copy of a MTHFD1 rs2236225 A allele (i.e., a A is present at nucleotide +1958 (coding region) of at least one copy of the MTHFD1 gene and the subject is administered the composition.

In some embodiments, the subject is a female. Further, in some embodiments, the subject is a premenopausal female and the determined genotype of the subject comprises at least one copy of a PEMT rs12325817 C allele, at least one copy of a MTHFD1 rs2236225 A allele, or combinations thereof and the subject is predicted to be susceptible to develop one or more choline deficiency-associated health effects and is therefore administered the therapeutic composition. Further, in some embodiments, the premenopausal female subject predicted to be susceptible to develop one or more choline deficiency-associated health effects is predicted to be susceptible to develop one or more congenital birth defects (e.g., neural tube defects) in a fetus carried by the subject as a result of choline deficiency in the subject and is therefore administered the therapeutic composition.

IV.A. Formulations

A therapeutic composition as described herein preferably comprises a composition that includes a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

IV.B. Doses

The term “effective amount” is used herein to refer to an amount of the therapeutic composition (e.g., a composition comprising choline, folate, or combinations thereof) sufficient to produce a measurable biological response (e.g., a reduction in one or more choline deficiency-associated health effects). Actual dosage levels of active ingredients in a therapeutic composition of the presently disclosed subject matter can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine. Minimal daily recommended dosages of choline (e.g., about 400-600 mg/day) and/or folate (e.g., about 300-500 μg/day) can be administered as an initial baseline to subjects being treated and slowly raised as necessary to maximum safe dosages.

For additional guidance regarding formulation and dose, see U.S. Pat. Nos. 5,326,902; 5,234,933; PCT International Publication No. WO 93/25521; Berkow et al. (1997); Goodman et al. (1996); Ebadi (1998); Katzunq (2001); Remington et al. (1975); Speight et al. (1997); and Duch et al. (1998).

IV.C. Routes of Administration

Suitable methods for administering to a subject a composition in accordance with the methods of the presently disclosed subject matter include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment.

The particular mode of drug administration used in accordance with the methods of the presently disclosed subject matter depends on various factors, including but not limited to the vector and/or drug carrier employed, the severity of the condition to be treated, and mechanisms for metabolism or removal of the drug following administration.

### V. SUBJECTS

A “subject” as the term is used herein generally refers to an animal. In some embodiments, an animal subject is a vertebrate subject. Further, in some embodiments, a vertebrate is warm-blooded and a representative warm-blooded vertebrate is a mammal. A representative mammal is most preferably a human. However, as used herein, the term “subject” includes both human and animal subjects. Thus, veterinary therapeutic uses are provided in accordance with the presently disclosed subject matter.

As such, the presently disclosed subject matter provides for the analysis and treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. A “subject” as the term is used herein can further include birds, such as for example those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, “subject” further includes livestock, including, but not limited to, domesticated swine, ruminants, ungulates, horses (including race horses), poultry, and the like.

Certain subjects are particularly vulnerable to choline deficiencies. In particular, subjects receiving an insufficient dietary intake of choline can be at risk of suffering from choline deficiency-associated health effects. For example, subjects receiving substantially all nutritional sustenance parenterally (e.g., via intravenous line) can be vulnerable to dietary choline deficiencies, which can result in for example liver dysfunction. Likewise, due to the increased nutrient demands on a mother by a fetus in utero, pregnant subjects can be vulnerable to choline deficiencies, which can result in choline deficiency-associated health effects in the pregnant subject and/or the fetus. As such, although not limited to these populations particular susceptible to dietary choline deficiencies, application of the presently disclosed subject matter methods to these subjects can provide significant benefits.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods for Examples 1-3

Subjects. Healthy adults were recruited by advertising. Both males (n=31) and females (n=31) were included, with ages ranging from 18 to 70 years. Inclusion was contingent on age-typical good state of health as determined by physical examination and standard clinical laboratory tests. Of the originally recruited 62 subjects, 58 completed at least the initial baseline phase and the depletion phase. Of these 58, 1 subject was excluded because of a 9-kg weight loss during the study, and 3 subjects were excluded because they did not comply with diet restrictions, leaving 54 subjects included in all analyses. Subject characteristics were as follows: 28 women and 26 men; 34 Caucasians, 14 African-Americans, 3 Asians, and 3 of other ethnicity; mean age was 38.7+/−15.4 (SD) years; mean body mass index was 25.0+/−3.7 kg/m². The ethnicity of the participants reflects the local population characteristics of the Raleigh-Durham-Chapel Hill area of North Carolina, U.S.A. The criteria for subject selection and all details of the clinical protocol were approved by the institutional review board of the University of North Carolina at Chapel Hill.

Clinical Studies.

The participants stayed at the University of North Carolina at Chapel Hill General Clinical Research Center, Chapel Hill, N.C., U.S.A. for the entire duration of the study and could leave only for brief periods under the direct supervision of study staff. All foods were prepared in-house to protocol specifications (Busby et al. (2004)). Total food intake was adjusted to be isocaloric and to provide adequate intake levels of macro- and micronutrients. Individual energy requirements were estimated by using the Harris-Benedict equation, and individual adjustments were made during the first week on the basal diet, if necessary, to achieve participants' satiety. Once individual needs had been determined, daily energy intakes were kept at a constant level, ranging between 35 and 45 kcal/kg of body weight. The diets, which provided 0.8 g/kg high biologic value protein, with 30% energy coming from fat and the remaining energy from carbohydrate, met or exceeded the estimated average requirement for methionine plus cysteine and the recommended dietary allowances for vitamin B12 and all other vitamins except folic acid (diets contained 100 μg/day folate, see below). During the initial 10 days (baseline), the participants consumed normal foods containing 550 mg of choline per 70 kg of body weight per day, which approximates the current adequate intake level (550 mg/day for men and 425 mg/day for women) and 400 μg of folic acid per day as a supplement (General Nutrition Center, Pittsburgh, Pa., U.S.A.). The actual choline content of a sampling of duplicate portions was assayed by our laboratory (Zeisel et al. (2003)).

Subjects were then switched to a choline depletion diet containing <50 mg of choline per 70 kg of body weight per day by eliminating choline-rich foods, as confirmed by analysis of duplicate food portions (Ubbink et al. (1991); Davis et al. (2005)). Details of diet formulations were previously published (Busby et al. (2004)). Table 2 provides an exemplary research diet menu including actual amounts of food provided for a 2500 kilocalorie diet containing varying amounts of choline. Furthermore, participants were randomly assigned to receive either placebo or 400 μg of folic acid per day as a supplement in addition to the amount of folate consumed with food (100 μg/day). Diets were well tolerated.

TABLE 2

| | Choline | | | | |
|---|---|---|---|---|---|
| | Deficient | Repletion Diets* | | | |
| | Diet | 25% | 50% | 75% | 100% |
| Food Item | Wt (g) | Wt (g) | Wt (g) | Wt (g) | Wt (g) |
| BREAKFAST | | | | | |
| Raw Egg White, Fresh | 100 | 50 | 50 | 50 | 50 |
| Wheat Starch/Lecithin | 20 | 20 | 40 | 65 | 85 |
| Bread** | | | | | |
| Margarine | 0 | 10 | 10 | 10 | 10 |
| Soy Protein Beverage | 300 | 240 | 240 | 0 | 0 |
| (Recipe II) | | | | | |
| Coffee, Decaf Instant | 2 | 2 | 2 | 2 | 2 |
| Cream Substitute- | 2 | 2 | 2 | 2 | 2 |
| Powder | | | | | |
| Coca Cola Classic, | 237 | 350 | 350 | 300 | 300 |
| Caffeine-Free | | | | | |
| AM SNACK | | | | | |
| Coca Cola Classic | 237 | 350 | 350 | 300 | 300 |
| LUNCH | | | | | |
| Cheese | 20 | 20 | 20 | 20 | 20 |
| Wheat Starch/Lecithin | 25 | 25 | 40 | 60 | 85 |
| Bread** | | | | | |
| Margarine | 15 | 15 | 15 | 15 | 10 |
| Applesauce, | 100 | 100 | 100 | 100 | 100 |
| unsweetened | | | | | |
| Soy Protein Beverage | 300 | 240 | 240 | 250 | 250 |
| (Recipe II) | | | | | |
| Coca Cola Classic, | 474 | 350 | 350 | 300 | 300 |
| Caffeine-Free | | | | | |
| PM SNACK | | | | | |
| Coca Cola Classic | 237 | 350 | 350 | 300 | 300 |
| DINNER | | | | | |
| Roasted Turkey | 20 | 20 | 20 | 20 | 20 |
| Breast, no skin | | | | | |
| French Fries, from | 30 | 30 | 30 | 30 | 30 |
| frozen | | | | | |
| Wheat Starch/Lecithin | 0 | 240 | 35 | 60 | 85 |
| Bread** | | | | | |
| Margarine | 0 | 0 | 0 | 15 | 15 |
| Soy Protein Beverage | 300 | 350 | 240 | 250 | 250 |
| (Recipe II) | | | | | |
| Coca Cola Classic | 237 | 0 | 350 | 300 | 300 |
| HS SNACK | | | | | |
| Tortilla Chips, Plain | 12 | 12 | 12 | 12 | 12 |
| Coca Cola Classic, | 237 | 350 | 350 | 300 | 250 |
| Caffeine-Free | | | | | |

Periodic determinations of urinary choline and betaine concentrations were used to confirm compliance with the dietary restrictions. Subjects remained on this depletion diet until they developed signs of organ dysfunction associated with choline deficiency, or for 42 days if they did not. Human subjects were deemed to have signs of organ dysfunction associated with choline deficiency if they had more than a 5-fold increase of serum creatine kinase (CK) activity while on the choline depletion diet and if this increased CK resolved when they were returned to the repletion diet (da Costa et al. (2004)), or if they had an increase of liver fat content by 28% or more while on the choline depletion diet and if this increased liver fat resolved when they were returned to the repletion or ad libitum diet (da Costa et al. (2005)). After the depletion period, subjects were repleted by gradually increasing choline intake and were maintained at a final level of >550 mg of choline per day for at least 3 days.

Change in liver fat content was estimated by MRI with a clinical MR system (VISION® 41.5-T, Siemens, Iselin, N.J., U.S.A.) using a modified “In and Out of Phase” procedure (da Costa et al. (2005); Fishbein et al. (1997)). This approach utilizes the differences in transverse magnetization intensity after an ultra brief time interval [fast low-angle shot (FLASH); echo time (TE)=2.2 msec and 4.5 msec, with a flip angle of 80°, and relaxation time (TR)_{—}140 msec]. Processing of successive FLASH MRI images with software from Siemens Medical Solutions (Malvern, Pa., U.S.A.) was used to estimate fat content. Organ content was derived from measurements across five liver slices per subject and standardized by relating the results to the similarly measured fat content of spleen. It was assumed that spleen signal would be largely invariant and used this value to calculate the outcome variable liver-to-spleen fat ratio.

Fasting blood samples were taken every 3-4 days throughout the study, and in particular, after 10 days on the 550 mg/day choline diet (baseline), at the end of the low-choline diet (depletion phase), and after consuming a repletion diet with 137-550 mg/day choline (repletion phase). The metabolic response to an oral challenge with 100 mg L-methionine/kg was determined initially and after depletion. Blood for homocysteine, SAM, and S-adenosylhomocysteine (SAH) measurements was obtained before and 4 hours after methionine ingestion (da Costa et al. (2005)).

Laboratory Analyses.

Plasma folate concentrations in fasted samples were measured by using a microbiological assay (Horne & Patterson (1988)). Serum was analyzed by using a dry-slide colorimetric method for CK activity by the McClendon Clinical Laboratories at University of North Carolina Hospitals, which is both Clinical Laboratory Improvement Act (CLIA)- and College of American Pathologists (CAP)-accredited. Total plasma homocysteine concentration was measured in fasted samples by using a HPLC method (da Costa et al. (2005); Ubbink et al. (1991)). SAM and SAH levels in plasma were measured by HPLC with fluorescence detection after conversion into their fluorescent isoindoles (Davis et al. (2005)).

Genotyping.

Genomic DNA was prepared according to manufacturer's instructions from peripheral blood with a commercial extraction kit (PUREGENE®, Gentra Systems, Minneapolis, Minn., U.S.A.) and diluted to a standard concentration of 1 μg/ml. The polymorphic sites of MTHFR (MTHFR-C677T and -A1298C), cytosolic C-1-THF synthase (MTHFD1-G1958A), and reduced folate carrier 1 (RFC1-G80A) were studied (see FIG. 2). The targeted DNA sequences were amplified by multiplex PCR, purified, and then analyzed with matrix-assisted laser desorption/ionization time-of-flight mass spectrometry (Meyer et al. (2004)). For all subjects, duplicate samples were genotyped. In the few instances of amplification failure, new DNA was prepared from backup blood samples.

Statistical Analysis.

Genotype-related differences in dichotomous outcomes were calculated with two-sided Fisher's exact probability test to determine statistical significance (GraphPad, San Diego, Calif., U.S.A.). Odds ratios for depletion by presence vs. absence of the predominant alleles were calculated as the odds to deplete for subjects with the allele divided by odds for subjects without the allele (Bland & Altman (2000)). Statistical significance of odds ratios was again calculated using two-sided Fisher's exact probability test. The statistical significance of group differences for continuous variables was assessed with Student's t test, and differences between subjects on different diets were assessed by using pair t tests. A two sample t test based on the differences between homocysteine concentrations in subjects on the two diets was used to compare the clinically depleted and not-depleted groups (Graph Pad).

### Example 1

### Genetic Variation and Folate Status

The distribution of the polymorphic variants of MTHFR and cytosolic MTHFD1 (Table 3) largely agreed with that of larger North European populations that were analyzed previously with the same genotyping methodology. Within this study group, however, fewer African-Americans than Caucasians had the variant allele MTHFD1 1958A (allele frequency 0.18 vs. 0.50). The RFC1 80G allele was slightly underrepresented in these subjects (0.47 vs. 0.58), but this was considered the reference allele, nonetheless. The difference is mostly attributable to the presence of many non-Caucasians in our regionally representative population sample.

Twenty-six participants were assigned to receive placebo, and 28 subjects received an additional 400 μg of folic acid per day as a supplement. Average serum folate concentration at the end of the depletion phase was lower in subjects with the lower folate intake (22.1+/−1.3 nmol_liter vs. 28.3+/−1.2 nmol/liter; P<0.01 by Student's t test). None of the investigated polymorphisms had a statistically significant effect on serum folate concentrations at any time point.

TABLE 3

| | | % subjects | | |
|---|---|---|---|---|
| | | with signs | | |
| | | of choline | | |
| Polymorphism | Genotype (n) | deficiency | P | Odds ratio and 95% CI |
| MTHFR 677 | CC (28) | 61 | 0.63 | CC vs. CT/TT |
| | CT (22) | 73 | | Odds ratio, 1.76 |
| | TT (4) | 75 | | 95% CI, 0.56-5.6 |
| MTHFR 1298 | AA (28) | 64 | 0.90 | AA vs AC/CC |
| | AC (22) | 68 | | Odds ratio, 1.25 |
| | CC (4) | 75 | | 95% CI, 0.40-3.9 |
| MTHFD1 1958 | GG (20) | 40 | 0.007 | GG vs GA/AA |
| | GA (28) | 82 | | Odds ratio, 7.0 |
| | AA (6) | 83 | | 95% CI, 2.0-25 |
| RFC1 80 | AA (19) | 56 | 0.59 | AA vs AG/GG |
| | AG (20) | 70 | | Odds ratio, 1.82 |
| | GG (15) | 73 | | 95% CI, 0.56-5.9 |

### Example 2

### Signs of Organ Dysfunction Associated with Choline Deficiency

Twelve subjects responded to the low-choline diet with an increase in serum CK activity, all but one within a month on the depletion diet. A significant increase in liver fat content was observed in another 24 participants, usually within a month on the depletion diet. In six of these participants, however, it took up to 42 days to accumulate the additional 28% or more of liver fat. Eighteen subjects did not show signs of organ dysfunction in response to the low-choline diet. Daily supplementation with folic acid did little to affect the likelihood of developing signs of choline deficiency [odds ratio, absence over presence of signs of choline deficiency in subjects with supplementation vs. without supplementation 0.8, 95% confidence interval (CI), 0.26-2.5].

### Example 3

### Methionine Metabolism

Homocysteine concentrations during both baseline and choline-depletion conditions were measured in 54 subjects, and methionine load tests were completed in 52 of these participants at the end of the baseline and depletion diet phases. Homocysteine concentration increased by 19% with the low-choline regime (P<0.001, paired t test). Supplementation with 400 μg/day folic acid blunted this increase, compared with placebo (15% vs. 23% increase, P<0.05, Student's t test). However, there was no significant interaction between fasting plasma homocysteine concentration and clinical status (P=0.113, Student's t test), because there was a similar increase of this measure in subjects judged to be clinically depleted (by 1.4 μmol/liter; 95% CI, 1.1-1.7) as in subjects without clinical signs of choline deficiency (0.9 μmol/liter; 95% CI, 0.5-1.4). None of the polymorphic variants had a statistically significant influence on plasma homocysteine concentrations, at either baseline or depletion. The expected rise of plasma homocysteine concentration after methionine loading was observed on both diets. The rise in homocysteine concentration in response to the methionine challenge in subjects eating the 550-mg choline diet was significantly less in subjects without the RFC1 80G allele than in the carriers of this allele (P=0.02, Student's t test). None of the other polymorphic variations were predictive for the metabolic response to methionine or the change of this response with choline depletion.

The rise in plasma homocysteine concentration after a methionine load was greater in individuals developing signs of choline deficiency when ingesting a low-choline diet than in those that did not. In this Example, after a methionine load at the end of the depletion phase, plasma homocysteine concentrations in the group with signs of choline deficiency rose 6.9 μmol/liter above that which was previously observed after a methionine load on the 550-mg choline diet (95% CI, 4.4-9.3, P_{—}0.0001). For subjects without signs of deficiency, plasma homocysteine did not increase significantly after the same methionine load (1.6 μmol/liter; 95% CI, −1.7-4.9, P=0.318).

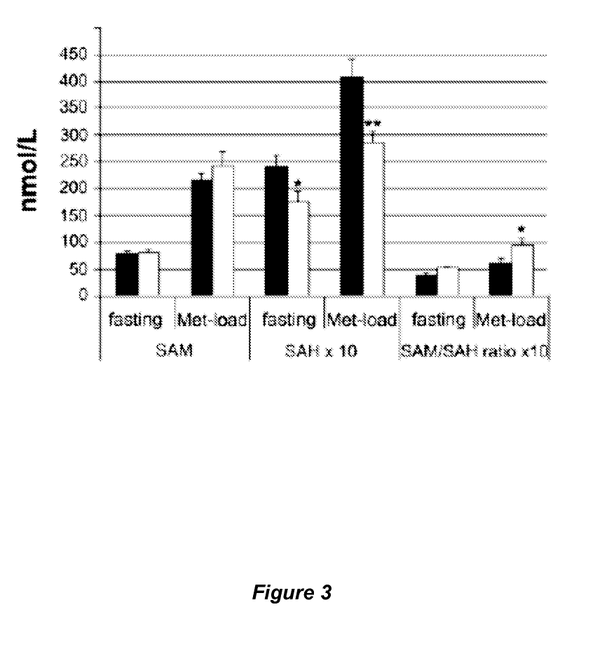

SAM and SAH concentrations were assessed in 26 individuals with MTHFD1 1958GA/AA genotype and in 15 individuals with MTHFD1 1958GG genotype. Concentrations did not change significantly upon switching from baseline to a low-choline diet. On both the baseline and the low-choline diets, SAM and SAH concentrations increased greatly after oral methionine load, as expected. The post-loading concentration of SAH increased from 28.8+/−12.8 nmol/liter on the baseline diet to 34.7+/−12.8 nmol/liter on the low-choline diet (P<0.05). No statistically significant change of post-loading SAM concentrations in response to the low-choline diet was observed. Although SAM concentrations at depletion did not differ significantly between MTHFD1 1958 genotype groups, SAH concentrations were significantly lower in participants with the GG genotype than in those with the GA and AA genotypes, both with and without methionine loading (FIG. 3). The same pattern of genotype-SAH concentration was observed while subjects were on the baseline diet, but the contrasts did not reach statistical significance.

Among the examined polymorphisms, the MTHFD1 G1958A variant was the best predictor of susceptibility to choline depletion (Table 3). In light of the subject numbers in several of the cells, the carriers of what are usually the minor alleles were grouped together for calculating odds ratios. Again, the MTHFD1 polymorphism was the only one of the four variants tested with a distinct impact on risk of developing clinical signs of choline deficiency. A higher percentage of the 34 carriers of the 1958A allele showed signs of choline deficiency in response to the low-choline diet (odds ratio, 7.0; two-sided, P=0.0025; Table 4). This genotypic difference was attributable to the fact that none of the young women with MTHFD1 1958GG genotype showed deficiency signs, whereas seven of the eight young women carriers of the GA or AA genotypes did so. The corresponding differences were not seen in men (odds ratio, 3.0; P=0.33) and postmenopausal women (odds ratio, 1.0; P=0.99). However, because only four postmenopausal women had the MTHFD1 1958-GG genotype, the power to detect an odds ratio of 7.0 (the average for all subjects) at the usual level of significance was only 0.13. With the eight male carriers of the GG genotype, the corresponding statistical power also was low. Thus, the study could have been underpowered for the detection of an effect smaller than the one observed in the young women it is possible effects measured could be significant given a study with a larger population.

In regard to folate supplementation, the MTHFD1 1958A allele-related difference in susceptibility to developing organ dysfunction when eating a low-choline diet was greatest in the group getting folate only from the diet (no supplement; odds ratio, 35; two-sided, P=0.001; Table 4) and was much smaller and statistically not significant in the folate-supplemented group (odds ratio, 2.5; two-sided, P=0.41; Table 4).

TABLE 4

| | | % subjects | | |
|---|---|---|---|---|
| | | with signs | | |
| | | of choline | | Odds ratio and |
| Group | Genotype (n) | deficiency | P | 95% CI |
| Premenopausal | GG (8) | 0 | | Odds ratio, 85* |
| women | GA/AA (8) | 88 | 0.000 | 95% CI, 3-2418 |
| Postmenopausal | GG (4) | 75 | | Odds ratio, 1.0 |
| women | GA/AA (8) | 75 | 0.99 | 95% CI, 0.06-16 |
| Men | GG (8) | 63 | | Odds ratio, 3.0 |
| | GA/AA (18) | 83 | 0.33 | 95% CI, 0.45-20 |
| All subjects | GG (20) | 40 | | Odds ratio, 7.0 |
| | GA/AA (34) | 82 | 0.007 | 95% CI, 2.0-25 |
| Diet folate only | GG (10) | 30 | | Odds ratio, 35 |
| | GA/AA (16) | 94 | 0.00 | 95% CI, 3.0-39 |
| Diet folate plus folic | GG (10) | 50 | | Odds ratio, 2.6 |
| acid supplement | GA/AA (18) | 72 | 0.41 | 95% CI, 0.52-13 |
| (400 μg/day) | | | | |

### Discussion of Examples 1-3

In the investigation of healthy subjects in Examples 1-3, more than half of the participants developed signs of organ dysfunction when consuming low-choline diets. Examples 1-3 focus on the impact of genetic variants of folate metabolism on susceptibility to clinical choline deficiency. One significant finding was the strong association of the MTHFD1 GI 958A polymorphism with susceptibility to developing signs of organ dysfunction associated with choline deficiency. Presence of the MTHFD1 1958A allele made it much more likely that subjects developed signs of choline deficiency.

Choline-deficient individuals were found to have impaired capacity to handle a methionine load, developing elevated plasma SAH and homocysteine concentrations. This finding highlights the importance of alternative folate-mediated pathways for homocysteine remethylation. The observation of higher SAH concentrations in carriers of the MTHFD1 1958A allele (rs2236225; SEQ ID NO:15), compared with non-carriers, is particularly informative, because accumulation of this metabolite has been found to be a more sensitive indicator of disturbed methionine regeneration than is elevated homocysteine concentration (Kerins et al. (2001)). SAH is a potent inhibitor of phosphatidylethanolamine methyltransferase, which catalyzes the endogenous formation of choline moiety in liver (Vance et al. (1997)).

Without wishing to be bound by any particular theory, phosphatidylethanolamine methyltransferase activity is increased by estrogen (Drouva et al. (1986)), and this mechanism could explain why it was observed that premenopausal women were relatively resistant to developing signs of organ dysfunction when fed a low-choline diet, compared with men. It is in these premenopausal women that the most significant effect of the MTHFD1 1958A SNP on susceptibility to developing signs of choline deficiency was observed (Table 4). Again, without wishing to be bound by theory, this suggests that this SNP restricts methyl-group availability enough so that SAM availability for the phosphatidylethanolamine methyltransferase-catalyzed formation of choline moiety becomes limiting, thereby eliminating this protective mechanism for females. The SAH data provided in these Examples (FIG. 3) support this hypothesis. Alternatively, it is possible that men and postmenopausal women are already so susceptible to choline deficiency (80% show signs of organ dysfunction on a low-choline diet) that a further increase in susceptibility cannot be appreciated, given the small incremental effect size. In premenopausal women, in contrast, where 60% of the population was resistant to choline deficiency, there was sufficient margin for detecting an increase in susceptibility associated with the MTHFD1 1958A SNP.

Under standard conditions, serine provides the bulk of one-carbon groups (Davis et al. (2004)). Cytosolic serine hydromethyltransferase (EC 2.1.2.1) transfers a one-carbon unit from serine to THF, and the resulting 5,10-methylene-THF can then be reduced by MTHFR to 5-methyl-THF. An alternative source for the one-carbon unit is derived from formate through mitochondrial or cytosolic reactions that can be linked to free folate by formyl-THF synthetase (EC 6.3.4.3) and generate 10-formyl-THF in an ATP-dependent reaction. This distinct reaction is only one of three that are catalyzed by the cytosolic enzyme C-1-THF synthase complex (all encoded by the MTHFD1 gene sequence). Two additional reactions, mediated by methylene-THF dehydrogenase (EC 1.5.1.5) and methenyl-THF cyclohydrolase (EC 3.5.4.9), can then convert 10-formyl-THF to 5,10-methylene-THF (FIG. 2). Although the formation of 5-methyl-THF is practically irreversible in vivo, the interconversion of 5,10-methylene-THF and 10-formyl-THF is closer to equilibrium (Home, D. W. (2003)). Thus, 5,10-methylene-THF can be directed either toward homocysteine remethylation or away from it. Both purine synthesis and oxidative release of carbon dioxide and THF by 10-formyl-THF dehydrogenase (EC 1.5.1.6) draw on the 10-formyl-THF pool. The irreversible and nonproductive dissipation of an excess in transferable one-carbon units is likely to be a significant regulatory factor, because the intrahepatic concentration of 10-formyl-THF exceeds the half-maximal equilibrium constant Km of 10-formyl-THF dehydrogenase (Gregory et al. (2000)). The 10-formyl-THF synthase activity of C-1-THF synthase, on the other hand, can add to the 5,10-methylene-THF pool by linking formate to free folate.

The G-to-A transition mutation at nucleotide 1958 in MTHFD1 causes an arginine to glutamine substitution in the protein region responsible for 10-formyl-THF dehydrogenase, which is far removed from the region providing for the methenyl-THF cyclohydrolase and methylene-THF dehydrogenase activities. Without wishing to be bound by theory, the MTHFD1 G1958A polymorphism could thus affect the delicately balanced flux between 5,10-methylene-THF and 10-formyl-THF and thereby influence the availability of 5-methyl-THF for homocysteine remethylation. The pattern of decreased SAM:SAH ratios among individuals with an MTHFD1 1958A allele appears to be consistent with the view that their one-carbon flux slightly tilts away from 5-methyl-THF formation. The finding of increased susceptibility to developing signs of choline deficiency coinciding with evidence of impaired 5-methyl-THF availability (increased SAH concentration) in carriers of an MTHFD1 1958A allele makes it less likely that the association is due to just random chance. If a nearby gene locus in strong linkage disequilibrium with the MTHFD1 1958A were ultimately responsible for increased susceptibility to choline deficiency, this locus also would have to explain the observed shift in methyl-group metabolism.

It is of particular interest that the gene-variant effect can be overcome if subjects are supplemented with folic acid. It might seem surprising that the development of choline deficiency signs was strongly favored by the presence of the MTHFD1 1958A allele but not by polymorphic variants of MTHFR or RFC1. A partial explanation could be provided by a recent investigation of folate-dependent homocysteine remethylation in young women (Davis et al. (2005)). This study found that the MTHFR 677TT genotype had little detectable effect on remethylation flux. In comparison, the MTHFD1 1958A polymorphism, which has not been extensively investigated until the present study, could be a more potent determinant of the rate at which one-carbon units become available for methyl-group transfer reactions, such as the synthesis of phosphatidylcholine from phosphatidylethanolamine.

Observations on genetically linked susceptibility to choline deficiency are important because they can assist refinement of recommendations for dietary choline intake by taking into account the needs of sizeable population groups with greater-than-average vulnerability to low choline or folate intake. There also is a potential relevance for the prevention of neural tube defects. One of the great successes of nutrition science has been the identification of the role that folate plays in normal neural tube closure; adequate dietary folate intake by mothers during pregnancy can prevent >50% of neural tube defects in babies (Shaw et al. (1995)).

As disclosed herein, choline and folate metabolism are highly interrelated (see FIG. 2). Inhibition of choline uptake and metabolism was associated with the development of neural tube defects in mice (Fisher et al. (2001); Fisher et al. (2002)). Recent evidence suggests that availability of choline also might impact the risk of neural tube defects in humans: A retrospective case-control study (400 cases and 400 controls) of periconceptional dietary intakes of choline in women found that women in the lowest quartile for daily choline intake had 4× the risk of having a baby with a neural tube defect than did women in the highest quartile for intake (Shaw et al. (2004)). The presently disclosed subject matter indicates the benefit of evaluating interactions among dietary choline intake, folate intake, and MTHFD1 polymorphisms.

### Materials and Methods for Example 4

Study Design.

Healthy males (n=31) and females (n=35) were recruited by advertising. They ranged in age from 18 to 70 years and had body mass indices between 19 and 33. Informed consent was obtained from all participants after the nature and possible consequences of the study were explained; the criteria for subject selection and all details of the clinical protocol were approved by the Institutional Review Board of the University of North Carolina at Chapel Hill (UNC-CH). The ethnicity of the participants was Caucasians (65%), African-Americans (25%), Asians (5%), Native Americans (3%), and other heritages (2%) reflecting the local population characteristics of the Raleigh-Durham-Chapel Hill area. Inclusion was contingent on age-typical good state of health as determined by physical examination and standard clinical laboratory tests. Of the originally recruited 66 subjects, 61 completed at least the initial phase and the depletion phase. Of these 61, 1 subject was excluded due to 9 kg wt loss during the study and 3 subjects were excluded because they did not comply with diet restrictions, leaving 57 subjects included in analyses.

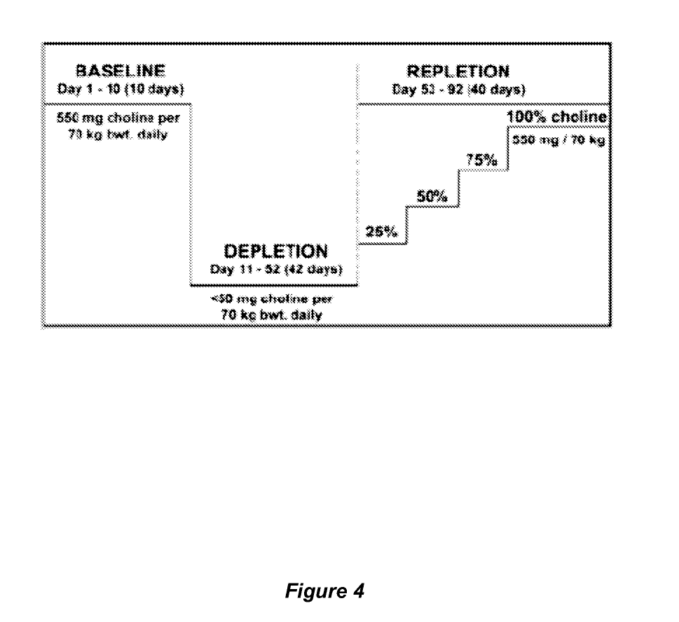

The participants were admitted to the UNC-CH General Clinical Research Center for the duration of the study and could leave only for brief periods under the supervision of study staff. The diets, which were composed of 0.8 g/kg high biological value protein, with 30% kcal coming from fat and the remaining kcal from carbohydrate, were prepared in-house to protocol specifications and are described in detail in another publication (Busby et al. (2004)). Total food intake was adjusted to be isocaloric and provided adequate intakes of macro- and micronutrients. Initially, all participants received a diet of normal foods containing 550 mg choline/70 kg body wt/day. This diet contained 50 mg betaine/70 kg body wt/day. After 10 days (FIG. 4) the choline content of the diet was reduced to <50 mg/day (with 6 mg betaine/70 kg body wt/day), as confirmed by analysis of duplicate food portions (Zeisel et al. (2003); Koc et al. (2002)). Periodic determinations of urinary choline and betaine concentrations (Koc et al. (2002)) were used to confirm compliance with the dietary restrictions. Subjects remained on this depletion diet until they developed organ dysfunction associated with choline deficiency, or for 42 days if they did not. Subjects were deemed to have organ dysfunction associated with choline deficiency if they had a >5-fold increase of serum creatine phosphokinase (CPK) activity (da Costa et al. (2004)) or if they had an increase in liver fat content by >28% while on the choline depletion diet, and if this increased CPK or increased liver fat resolved when choline was returned to the diet. After the depletion study, subjects were repleted by gradually increasing their choline intake to a final concentration of >550 mg per day and maintaining that concentration for at least 3 days. All 57 (54 for PEMT rs7946 (SEQ ID NO:3) and BHMT rs3733890 (SEQ ID NO:26) individuals were genotyped as described below for the following SNPs (the positions of the PEMT SNPs were enumerated with promoter B (Shields et al. (2001)) as a reference): PEMT rs7946 (G5465A; SEQ ID NO:3), rs2278952 (C164T; SEQ ID NO:2), rs12325817 (G-744C; SEQ ID NO:1) and for two other SNPs of PEMT at position C-314T of the PEMT promoter, and C29G of exon 2, which adjoins the promoter; BHMT rs3733890 (SEQ ID NO:26); CHDH rs9001 (SEQ ID NO:13), and rs12676 (SEQ ID NO:14) (Table 5). Plasma collected at the end of the adequate choline intake phase and depletion phase were analyzed for concentrations of betaine, choline, and phosphatidylcholine (Koc et al. (2002)).

Liver Fat Measurement.

Liver fat was measured at the end of the 550 mg choline diet and at 21 and 42 days on the choline-deficient diet. Change in liver fat content was estimated by MRI with a Siemens VISION® 41.5T clinical MR system using a modified “In and Out of Phase” procedure (da Costa et al. (2005); Fishbein et al. (1997)). This approach utilizes the differences in transverse magnetization intensity after an ultra-brief time interval (FLASH; TE=2.2 ms and 4.5 ms, with a flip angle of 80°, and TR=140 ms). Processing of successive FLASH MRI images with software from Siemens Medical Solutions (Malvern, Pa., U.S.A.) was used to estimate fat content. Organ fat content was derived from measurements across 3-5 liver slices per subject and standardized by relating the results to the fat content of similarly measured slices of spleen.

Laboratory Analyses.

Fasting blood samples were taken every 3-4 days for blood chemistries (including CPK analysis), after 10 days on the 550 mg/day choline diet (baseline), and at the end of the low choline diet (depletion phase) for choline and genotyping studies. Serum was analyzed using a dry slide colorimetric method for CPK activity by the McClendon Clinical Laboratories at University of North Carolina Hospitals, which is both Clinical Laboratory Improvement Act and College of American Pathologists accredited. Choline and its metabolites were analyzed and quantified directly by HPLC mass spectrometry (LC/ESI-IDMS) after the addition of internal standards labeled with stable isotopes that were used to correct for recovery (Koc et al. (2002)).

Genotyping. Peripheral lymphocytes were isolated from blood by Ficoll-Hypaque gradient using VACUTAINER® CPT™ tubes with sodium citrate (Becton Dickinson, Franklin Lakes, N.J., U.S.A.) (Fotino et al. (1971); Ting & Morris et al. (1971)) and genomic DNA extracted using PUREGENE® (Gentra Systems, Minneapolis, Minn., U.S.A.) according to the manufacturer's instructions. SNP analyses were carried out as described below. Table 5 lists SNPs studied for this Example. Briefly, for the PEMT and CHDH genes, DNA sequencing was performed on double-stranded DNA templates obtained from genomic DNA by polymerase chain reaction (PCR) amplification. A negative control without DNA and a positive control with human DNA (PROMEGA Inc., Madison, Wis., U.S.A.) for each PCR set were included. PCR products were purified with QIAQUICK® PCR Purification Kit 250 (QIAGEN Inc., Valencia, Calif., U.S.A.) after electrophoresis in 0.8% or 3% agarose depending on the size of the fragment. Sequencing reactions were performed by the University of North Carolina at Chapel Hill Genome Analysis Facility, using a capillary sequencing machine (model 3100, Applied Biosystems, Foster City, Calif., U.S.A.). Sequence results were interpreted using the program SEQUENCHER™ (Gene Code Corp., Ann Arbor, Mich., U.S.A.). Basic local alignment search tool searches were performed using the National Center for Biotechnology (NCBI) program available through the NCBI website.

TABLE 5

| | | Base pair and sequence |
|---|---|---|
| Gene | rs number | change |
| MTHFD1 | rs2236225 | +1958 G → A |
| PEMT | rs12325817 | −744 G → C^{b} |
| PEMT | rs2278952 | +164 C → T^{b} |
| PEMT | rs7946 | +5465 G → A^{b} |
| PEMT | not yet designated | −314 C→T^{b} |
| PEMT | not yet designated | +29 C→G^{b} |
| CHDH | rs9001 | +318 A → C |
| CHDH | rs12676 | +432 G → T |
| BHMT | rs3733890 | +742 G → A |

Promoter of the PEMT Gene.

Successful amplification of the 1896 bp DNA fragment of the PEMT gene was performed using TAKARA™ Ex Taq polymerase (Fisher Scientific, Fair Lawn, N.J., U.S.A.) with an efficient 3′-5′ exonuclease activity for increased fidelity. Based on the GENBANK® sequence (accession number NC_{—}000017), a set of primers for amplification was designed, as recommended by the manufacturers, and a set of primers for sequencing the overlapping segments in two directions (Table 6) using the WEB PRIMERS™ design program available through the Stanford University website. The forward and reverse primers were 5′GAGCACGTGAGCTGTCAGTGCCTTTTG3′ (SEQ ID NO:30) and 5′CCAACCTCCTTCATACAACAGAGGTCC3′ (SEQ ID NO:31), respectively, and a three-step PCR was performed on an Applied Biosystems 2720 thermal cycler (Foster City, Calif., U.S.A.) under the following conditions: 96° C. for 2 min; 30 cycles (94° C. for 30 s, 60° C. for 1 min, 72° C. for 2 min); extend 72° C. for 7 min, and soak at 4° C. For the sequence determination of PEMT rs12325817 (SEQ ID NO:1), an additional primer was used (PEMT PRO seq-F2 (SEQ ID NO:35; Table 6) to verify the sequence in a region containing Alu repeats and a poly-A tail.

TABLE 6

| Position of primer | | |
|---|---|---|
| (bp) | Primer name | Sequence of primer 5′-3′ |
| 7728-7757 | PEMT PRO-F3 | GGAGTTATGGATCTAGGGAACTGGAGCAGC |
| | | (SEQ ID NO: 32) |
| 7711-7730 | PEMT PROseq-F1 | ATTTCACCCTCCTGAAAGGA |
| | | (SEQ ID NO: 33) |
| 8102-8082 | PEMT PROseq-R1 | TGACCAATCTAAGCCCAGGTT |
| | | (SEQ ID NO: 34) |
| 8092-8112 | PEMT PROseq-F2 | TAGATTGGTCATGGGAGGCTT |
| | | (SEQ ID NO: 35) |
| 8493-8474 | PEMT PROseq-R2 | ACAACATGGTGACACTCCGT |
| | | (SEQ ID NO: 36) |
| 8503-8522 | PEMT PROseq-F3 | TCTCGAACTCCTGACCATCA |
| | | (SEQ ID NO: 37) |
| 8878-8860 | PEMT PROseq-R3 | CCCGTAATCCCAGCACTTT |
| | | (SEQ ID NO: 38) |
| 8915-8935 | PEMT PROseq-F4 | GAGGAAAAAGACTCTGGCACA |
| | | (SEQ ID NO: 39) |
| 9300-9280 | PEMT PROseq-R4 | TTTACTCCATTGAGGGGTGCA |
| | | (SEQ ID NO: 40) |
| 9084-9101 | PEMT PROseq-F5 | TGATGGATCCCAGGAGGA |
| | | (SEQ ID NO: 41) |
| 9510-9490 | PEMT PROseq-R5 | GGCTTTCTGCTACCCAGTAAT |
| | | (SEQ ID NO: 42) |
| 9525-9498 | PEMT PRO-R1 | ACAACAGAGGTCCCGGCTTTCTGCTAC |
| | | (SEQ ID NO: 43) |
| 8438-8458 | PEMT PROseqMid-F3 | ACAACAGAGGTCCCGGCTTTCTGCTAC |
| | | (SEQ ID NO: 44) |
| 8824-8802 | PEMT PROseqMid-R3 | TCAGAGATCAGCCTGGCCAATAT |
| | | (SEQ ID NO: 45) |
| 8461-8480 | PEMT PROseqMid-F4 | ATTTTTAGTAGAGACGGAGT |
| | | (SEQ ID NO: 46) |
| 8840-8821 | PEMT PROseqMid-R4 | ATCACAAGGCCAGGAGTCAG |
| | | (SEQ ID NO: 47) |
| 8374-8394 | PEMT PRO SNP1-F | ACTTCCTGGGTTGAAGCGATT |
| | | (SEQ ID NO: 48) |
| 8597-8579 | PEMT PRO SNP1-R | TTTATTCTCTGGCCGTGCC |
| | | (SEQ ID NO: 49) |

PEMT.

The coding region of PEMT containing the SNP rs7946 was amplified with the oligonucleotides 5′GGAGCACTTTGCCCCAGAATC3′ (SEQ ID NO:50) and 5′GACTTGGAGCCTTCAGAGCG3′ (SEQ ID NO:51) as forward and reverse primers, respectively (Song et al. (2005)). The sequences obtained were compared with ones stored in the NCBI database available through the NCBI website (accession number AF294467) using ClustalW multiple sequence alignment software available through the European Bioinformatics Institute website.

CHDH.

Amplification of the 370 bp DNA fragment containing SNPs rs9001 (SEQ ID NO:13) and rs12676 (SEQ ID NO:14) in the CHDH gene was performed using BD ADVANTAGE™ GC Genomic PCR Kit (BD Biosciences, Mountain View, Calif., U.S.A.). Based on the GENBANK® sequence (accession number NC_{—}000003), the following primers were designed for amplification and sequencing: 5′AGTCATCTCATTCCCCTCCGTGGATCAGA3′ (forward primer; SEQ ID NO:52) and 5′TAGCACCAGTTGTACCTGTCGTCGCACA3′ (reverse primer; SEQ ID NO:53). A two-step PCR was done with the following conditions: 94° C. for 1 min; 30 cycles (94° C. for 30 s, 68° C. for 3 min); extend 70° C. for 5 min and soak at 15° C. The SNPs were numbered with the mRNA location (318 and 432, respectively; NT_{—}018397).

BHMT.

For the variant rs3733890 (SEQ ID NO:26) in exon 6 of BHMT (GENBANK® accession number NT_{—}006713), the targeted DNA sequence: 5′GCCACTTTGACCCCACCATTAGT3′ (SEQ ID NO:54) and 5′TGGGAATTCTGGGAGATCGATG3′ (SEQ ID NO:55) as forward and reverse primers, respectively, were amplified by multiplex PCR, purified, then analyzed with matrix-assisted laser desorption/ionization time-of-flight mass spectrometry (Meyer al. (2004)). Samples were analyzed in duplicate.

Statistical Analysis.

Data for continuous variables are expressed as mean+/−SE, and the statistical significance of differences between subjects on the two different diets were assessed using paired t test. A two-sample t test based on the differences between choline and metabolite concentrations in subjects on the baseline and depletion diets was used to compare the depleted with organ dysfunction and depleted without organ dysfunction groups. Genotype differences associated with organ dysfunction associated with choline deficiency were calculated using Fisher's Exact Test to determine statistical significance (Lowry (2004). For P<0.05, odds ratios and 95% confidence intervals were calculated as the odds of showing signs of deficiency for subjects with the risk allele divided by the odds of showing signs of deficiency for subjects without the risk allele. The Kruskal-Wallis test was used to compare differences in continuous variables by genotype (Kruskal & Wallis (1952)).

### Example 4

Of the 57 participants, 68% developed organ dysfunction when fed the low choline diet and this resolved when choline was added back to their diets. Plasma betaine concentrations decreased almost 50% in response to the low choline diet (from 60+/−3 to 32+/−2 nmol/ml; P<0.001) and choline concentrations decreased almost 30% (from 9.8+/−0.3 to 7.1+/−0.2 nmol/ml; P<0.001). These decreases were irrespective of whether or not subjects developed organ dysfunction on the low choline diet. Plasma phosphatidylcholine concentrations were 9% lower when subjects were fed the low choline diet (1691+/−41 vs. 1868+/−45 nmol/ml than when on baseline diet; P<0.001); those subjects who developed organ dysfunction on the low choline diet had a 3-fold greater decrease in phosphatidylcholine (−228+/−47 nmol/ml) than those who did not (−64+/−31 nmol/ml; P<0.029 by t test). Plasma choline and phosphatidylcholine concentrations did not differ from baseline after subjects were repleted with choline-containing diets. Significant changes in choline, betaine, or phosphatidylcholine concentrations were not detected in plasma between genotypes (note that there was no significant change in plasma betaine concentrations associated with the BHMT genotype tested).

Gender was one modifier of susceptibility to developing organ dysfunction when fed a low choline diet. When deprived of dietary choline, 77% of men and 80% of postmenopausal women developed fatty liver or muscle damage, whereas only 44% of premenopausal women developed such signs of organ dysfunction associated with choline deficiency. Note that within each gender grouping, a significant number of subjects were resistant to developing organ dysfunction, suggesting that other factors, such as genetic polymorphisms, contribute to susceptibility to developing organ dysfunction when fed a low choline diet.

For each SNP, allelic association was tested for correlation with susceptibility to developing organ dysfunction associated with eating a low choline diet. Two SNPs in the PEMT promoter region were identified and tested: rs12325817 (G-774C; SEQ ID NO:1) and an SNP at position C-314T. A SNP in exon 4 (G54653; rs7946; SEQ ID NO:3) and two SNPs in exon 2, which adjoined the promoter, rs2278952 (C164T; SEQ ID NO:2) and C29G, were found and tested as well. For rs12325817 (G-774C; SEQ ID NO:1), the variant C allele was relatively common in our study population, where 18% were CC, 56% GC, and 26% GG genotype. The SNPs at position −314 in the PEMT promoter and +29 of exon 2 were rare, each occurring as a heterozygous allele in one subject (0.032 frequency). Neither developed organ dysfunction when on the low choline diet. The rs2278952 SNP (C164T; SEQ ID NO:2) occurred at a frequency of 0.25 in women. It, too, was a heterozygous allele and was not associated with changes in susceptibility to developing organ dysfunction when on the low choline diet.

In all women, 18 of the 23 (78%) carriers of the PEMT-744C allele (rs12325817; SEQ ID NO:1) developed organ dysfunction when fed a low choline diet (odds ratio 25, P=0.002; Table 7). In postmenopausal women, 11 of 12 (92%) of the allele carriers developed organ dysfunction when fed a low choline diet, and the two women without this allele did not. In the eight premenopausal women who were heterozygous for the allele (GC genotype), half developed organ dysfunction and half did not when fed a low choline diet, while the two premenopausal women who were homozygous for the allele developed organ dysfunction. Overall, the three women homozygous for this allele (CC genotype) developed organ dysfunction when fed a low choline diet, and seven of the eight females without this allele (GG genotype) did not (Table 7). There was no effect in men.

TABLE 7

| Signs of | | | | | p value |
|---|---|---|---|---|---|
| choline | | | | | OR (95% |
| deficiency | | GG | GC | CC | CI) |
| All subjects (57) | Yes | 7 | 25 | 7 | 0.10 |
| | No | 8 | 7 | 3 | |
| Men (26) | Yes | 6 | 10 | 4 | 0.49 |
| | No | 1 | 2 | 3 | |
| All women (31) | Yes | 1 | 15 | 3 | 0.002 |
| | No | 7 | 5 | 0 | 25 (2, 256) |
| Pre menopausal (16) | Yes | 1 | 4 | 2 | 0.10 |
| | No | 5 | 4 | 0 | |
| Postmenopausal (15) | Yes | 0 | 11 | 1 | 0.03 |
| | No | 2 | 1 | 0 | 42 (1, |
| | | | | | 1348)* |

The first of two SNPs in the coding region of the CHDH gene (rs9001; A318C; SEQ ID NO:13) had a protective effect on susceptibility to developing organ dysfunction when fed a low choline diet in all subjects who carried the C allele (Table 8). No significant differences were found when the participants were grouped by gender or menopausal status. Although the second CHDH variant (rs12676; G432T; SEQ ID NO:14) was within 115 base pairs of SNP rs9001 (A318C; SEQ ID NO:13), it was formed independently (no difference in Hardy-Weinberg expected distribution for the population). Among all subjects, this SNP was not associated with susceptibility to developing organ dysfunction associated with choline deficiency. However, among premenopausal women, 5 of the 6 (83%) who were heterozygous for this variant developed organ dysfunction on a low choline diet compared with 2 of 10 (20%) who did so without this risk allele (odds ratio 20, P=0.04; Table 8).

TABLE 8

| | Signs | CHDH A318C | | | | CHDH G432T | | | |
|---|---|---|---|---|---|---|---|---|---|
| | of | rs9001 | | | | rs12676 | | | |
| | choline | | | | p value | | | | p value |
| | defi- | | | | OR | | | | OR |
| | ciency | AA | AC | CC | (95% CI) | GG | GT | TT | (95% CI) |
| All | Yes | 34 | 4 | 1 | 0.03 | 19 | 19 | 1 | 0.23 |
| subjects | No | 10 | 6 | 2 | 0.2 | 13 | 5 | 0 | |
| (57) | | | | | (0.05, 0.7) | | | | |
| Men | Yes | 18 | 2 | 0 | 0.22 | 10 | 9 | 1 | 1.00 |
| (26) | No | 4 | 2 | 0 | | 3 | 3 | 0 | |
| All | Yes | 16 | 2 | 1 | 0.13 | 9 | 10 | 0 | 0.07 |
| women | No | 6 | 4 | 2 | | 10 | 2 | 0 | |
| (31) | | | | | | | | | |
| Pre- | Yes | 6 | 1 | 0 | 0.39 | 2 | 5 | 0 | 0.04 |
| menopausal | No | 4 | 3 | 2 | | 8 | 1 | 0 | 20 |
| (16) | | | | | | | | | (1, 282) |
| Post- | Yes | 10 | 1 | 1 | 0.52 | 7 | 5 | 0 | 1.00 |
| menopausal | No | 2 | 1 | 0 | | 2 | 1 | 0 | |
| (15) | | | | | | | | | |

There was no association between the SNP tested in exon 4 of the PEMT gene (rs7946, G5465A; SEQ ID NO:3) and susceptibility to choline deficiency (Table 9), nor was the BHMT variant (rs3733890; G742A; SEQ ID NO:26) associated with changes in susceptibility to choline deficiency (Table 9).

TABLE 9

| | Signs | | | | | | | | |
|---|---|---|---|---|---|---|---|---|---|
| | of | PEMT rs7946, | | | | BHMT G742A | | | |
| | choline | G5465A | | | | rs3733890 | | | |
| | defi- | | | | p | | | | p |
| | ciency | GG | GA | AA | value | GG | GA | AA | value |
| All subjects | Yes | 5 | 16 | 15 | 0.86 | 21 | 11 | 4 | 0.84 |
| (54) | No | 3 | 9 | 6 | | 9 | 7 | 2 | |
| Men | Yes | 4 | 8 | 8 | 1.00 | 12 | 6 | 2 | 1.00 |
| (26) | No | 1 | 3 | 2 | | 4 | 1 | 1 | |
| All women | Yes | 1 | 8 | 7 | 0.75 | 9 | 5 | 2 | 0.65 |
| (28) | No | 2 | 6 | 4 | | 5 | 6 | 1 | |
| Pre- | Yes | 0 | 5 | 2 | 0.63 | 4 | 2 | 1 | 0.79 |
| menopausal | No | 2 | 4 | 3 | | 3 | 5 | 1 | |
| (16) | | | | | | | | | |
| Post- | Yes | 1 | 3 | 5 | 0.66 | 5 | 3 | 1 | 1.00 |
| menopausal | No | 0 | 2 | 1 | | 2 | 1 | 0 | |
| (12) | | | | | | | | | |

### Discussion for Example 4

Common genetic polymorphisms have been reported to influence human requirements for nutrients. For example, a common SNP in the methyltetrahydrofolate reductase gene increases dietary requirements for the vitamin folic acid (Shelnutt et al. (2003)). However, these SNPs usually have very modest effects on nutrient needs. As disclosed hereinabove in Examples 1-3, it was determined that individuals who were carriers of the MTHFD11958A allele were more likely than non-carriers to develop signs of choline deficiency. The present Example discloses the identification of genetic variations in the PEMT and CHDH genes that are associated with developing organ dysfunction when choline is removed from the diet of subjects. These polymorphisms influence the susceptibility of developing organ dysfunction in subjects when fed a low choline diet, and thus they increase the dietary requirement for choline needed to sustain optimal health in subjects carrying these alleles.

In particular, women with a common variant in the promoter region of the PEMT gene rs12325817 (G −774C; SEQ ID NO:1) were at significantly increased risk of developing organ dysfunction when dietary intake of choline is insufficient. PEMT activity is responsible for endogenous biosynthesis of choline moiety (Zeisel & Blusztajn (1994)), and this activity is increased by estrogen treatment (36). The promoter region of this gene is likely to have an estrogen response element (ERE). Indeed, the rs12325817 (G-774C; SEQ ID NO:1) SNP is located within 50 bp of a putative ERE, which contains a perfect half-site consensus sequence, but four of five bases differ in the other half-site. Without wishing to be bound by any particular theory, given the sexually dimorphic effect of PEMT rs12325817 (G-774C; SEQ ID NO:1), it is possible that this SNP alters the estrogen responsiveness of the promoter. Again, without wishing to be bound by theory, premenopausal women who are heterozygous for the PEMT rs12325817 (G-774C) C allele likely have sufficient estrogen to overcome the effects on estrogen-mediated transcription factor of the single allele, whereas postmenopausal women with lower estrogen levels are sensitive to the SNP. Men, with little estrogen, would be unaffected by an SNP that altered estrogen receptor complex binding.

A protective effect against choline deficiency of the SNP in the CHDH gene (rs9001; A318C; SEQ ID NO:13) was determined, even though the frequency of this allele was relatively low (0.23). A significant decrease in susceptibility to developing organ dysfunction on a low choline diet in all subjects was found. A correlation with sensitivity to choline deficiency was also found with the CHDH rs12676 (G432T; SEQ ID NO:14) SNP.

The lack of effect of the SNP tested in exon 4 of the PEMT gene (rs7946, G5465A) was unexpected, because it was previously reported that this is a loss of function SNP and that persons with the variant A allele have increased risk of nonalcoholic fatty liver disease (Song et al. (2005)). Perhaps the modest decrease (30%) in activity of PEMT associated with this SNP was overshadowed by compensatory induction of the enzyme that is associated with choline deficiency in males (Cui & Vance (1996); Johnson & Blusztajn (1998)) and by estrogen-mediated activation of PEMT in females. With regard to the BHMTSNP effect, the protein product of the gene variant did not differ in either catalytic activity or betaine binding when compared to the enzyme which did not contain the polymorphism.

The SNPs identified and disclosed herein that increased susceptibility to developing organ dysfunction in human subjects fed low choline diets are believed to be of clinical importance. Humans fed intravenously (total parenteral nutrition) with solutions that deliver less choline than the adequate intake concentration often develop liver dysfunction that sometimes resolves when a choline source is added to their feeding solution (Buchman et al. (2001)). Subjects carrying the identified SNPs are the ones most likely to be susceptible to this complication of parenteral nutrition.

These SNPs, combined with poor dietary intake of choline, can contribute to adverse outcomes during pregnancy, a time when choline demand is high (Shaw et al. (2004); Zeisel et al. (1995)). Deficient maternal dietary intake of choline during pregnancy in humans has been associated with a 4-fold increased risk of having a baby with a neural tube defect (Shaw et al. (2004)). In rodent models, maternal dietary choline intake influenced brain development. More choline (4 dietary levels) during days 11-17 of gestation in the rodent increased hippocampal progenitor cell proliferation (Albright et al. (1999a); Albright et al. (1999b)), decreased apoptosis in these cells (Albright et al. (1999a); Albright et al. (1999b)), enhanced long-term potentiation (LTP) in the offspring when they were adult animals (Jones et al. (1999); Pyapali et al. (1998); Montoya et al. (2000)), and enhanced visuospatial and auditory memory by as much as 30% in the adult animals throughout their lifetime (Meck & Williams (1999); Meck & Williams (1997a); Meck & Williams (1997b); Meck & Williams (1997c); Meck et al. (1998); Meck & Williams (2003); Williams et al. (1998)). Mothers fed choline-deficient diets during late pregnancy have offspring with diminished progenitor cell proliferation and increased apoptosis in fetal hippocampus (Albright et al. (1999a); Albright et al. (1999b)), insensitivity to LTP when they were adult animals (Jones et al. (1999)), and decremented visuospatial and auditory memory (Meck & Williams (1999)). For these reasons, identification of common polymorphisms that increase dietary requirements for choline during pregnancy, such as for example those disclosed herein, enables the identification of women for whom adequate dietary choline intake should be assured.

In summary, it is disclosed in the present Example for the first time that SNPs in the phosphatidylethanolamine N-methyltransferase (PEMT) and choline dehydrogenase (CHDH) genes are associated with altered susceptibility to developing organ dysfunction on a low choline diet, and they affect dietary requirements for the nutrient choline. These SNPs are common, and their effects should be considered when setting dietary reference intake levels. In addition, since the genes of interest have many more polymorphisms than were specifically tested, unmeasured but causal genetic variation can be in linkage disequilibrium with the exemplary SNPs specifically genotyped. As such, the presently disclosed subject matter is intended to be inclusive of all choline metabolism gene polymorphisms correlated with choline deficiency-associated health effects, including those in linkage disequilibrium with polymorphisms exhibiting direct effects on peptide function.

### REFERENCES

All references cited in the specification, including but not limited to U.S. and foreign patents and patent application publications, scientific journal articles, and database entries (including all annotations presented therein), are incorporated herein by reference to the extent that they supplement, explain, provide a background for or teach methodology, techniques and/or compositions employed herein.
Aarsaether, N., Berge, R. K., Aarsland, A., Svardal, A. & Ueland, P. M. (1988) Biochim. Biophys. Acta 958, 70-80.
Albright, C. D., Lui, R., Bethea, T. C., da Costa, K.-A., Salganik, R. I. & Zeisel, S. H. (1996) FASEB J. 10, 510-516.
Albright, C. D., Friedrich, C. B., Brown, E. C., Mar, M. H., and Zeisel, S. H. (1999a) Brain Res. Dev. 115, 123-129.
Albright, C. D., Tsai, A. Y., Friedrich, C. B., Mar, M. H., and Zeisel, S. H. (1999b) Brain Res. Dev. 113, 13-20.
Albright, C. D., da Costa, K.-A., Craciunescu, C. N., Klem, E., Mar, M. H. & Zeisel, S. H. (2005) Cell. Physiol. Biochem. 15, 59-68.
Bailey, L. B. & Gregory, J. F., III (1999) J. Nutr. 129, 779-782.
Berkow et al., (1997) The Merck Manual of Medical Information, Home ed. Merck Research Laboratories, Whitehouse Station, N.J.
Bland, J. M. & Altman, D. G. (2000) Br. Med. J. 320, 1468.
Blusztajn, J. K., Zeisel, S. H. & Wurtman, R. J. (1985) Biochem. J. 232, 505-511.
Brody, L. C., Conley, M., Cox, C., Kirke, P. N., McKeever, M. P., Mills, J. L., Molloy, A. M., O'Leary, V. B., Parle-McDermott, A., Scott, J. M. & Swanson, D. A. (2002) Am. J. Hum. Genet. 71, 1207-1215.
Buchman, A. L., Ament, M. E., Sohel, M., Dubin, M., Jenden, D. J., Roch, M., Pownall, H., Farley, W., Awal, M., and Ahn, C. (2001) J. Parenter. Enteral Nutr. 25, 260-268.
Buchman, A., Dubin, M., Moukarzel, A., Jenden, D., Roch, M., Rice, K.,
Busby, M. G., Fischer, L., Da Costa, K. A., Thompson, D., Mar, M. H., and Zeisel, S. H. (2004) J. Am. Diet Assoc. 104, 1836-1845.
Cui, Z., and Vance, D. E. (1996) J. Biol. Chem. 271, 2839 -2843.
da Costa, K. A., Badea, M., Fischer, L. M., and Zeisel, S. H. (2004) Am. J. Clin. Nutr. 80, 163-170.
da Costa, K. A., Gaffney, C. E., Fischer, L. M., and Zeisel, S. H. (2005) Am. J. Clin. Nutr. 81, 440-444.
Davis, S. R., Stacpoole, P. W., Williamson, J., Kick, L. S., Quinlivan, E. P., Coats, B. S., Shane, B., Bailey, L. B. & Gregory, J. F., III (2004) Am. J. Physiol. 286, E272-E279.
Davis, S. R., Quinlivan, E. P., Shelnutt, K. P., Maneval, D. R., Ghandour, H., Capdevila, A., Coats, B. S., Wagner, C., Selhub, J., Bailey, L. B., et al. (2005a) J. Nutr. 135, 1040-1044.
Davis, S. R., Quinlivan, E. P., Shelnutt, K. P., Ghandour, H., Capdevila, A., Coats, B. S., Wagner, C., Shane, B., Selhub, J., Bailey, L. B., et al. (2005b) J. Nutr. 135, 1045-1050.
Drouva, S. V., LaPlante, E., Leblanc, P., Bechet, J. J., Clauser, H. & Kordon, C. (1986) Endocrinol. 119, 2611-2622.
Duch et al., (1998) Toxicol. Lett. 100-101:255-263.
Ebadi, (1998) CRC Desk Reference of Clinical Pharmacology. CRC Press, Boca Raton, Fla.
Fishbein, M., Gardner, K., Potter, C., Schmalbrock, P., and Smith, M. (1997) Magn. Reson. Imaging 15, 287-293.
Fisher, M. C., Zeisel, S. H., Mar, M. H. & Sadler, T. W. (2001) Teratology 64, 114-122.
Fisher, M. C., Zeisel, S. H., Mar, M. H. & Sadler, T. W. (2002) FASEB J. 16, 619-621.
Fotino, M., Merson, E., and Allen, F. (1971) Ann. Clin. Lab. Sci. 1, 131-133.
Freeman-Narrod, M., Narrod, S. A. & Yarbro, J. W. (1977) Med. Pediatr.
Freireich et al., (1966) Cancer Chemother Rep. 50:219-244.
Genest, J., Jr., Mar, M. H., Zeisel, S. H., Castro, C., Garrow, T. & Rozen, R.
Goodman et al., (1996) Goodman & Gilman's the Pharmacological Basis of Therapeutics, 9th ed. McGraw-Hill Health Professions Division, New York.
Gornbein, J. & Ament, M. (1995) Hepatology 22, 1399-1403.
Gregory, J. F., III, Cuskelly, G. J., Shane, B., Toth, J. P., Baumgartner, T. G. & Stacpoole, P. W. (2000) Am. J. Clin. Nutr. 72, 1535-1541.
Horne, D. W. & Patterson, D. (1988) Clin. Chem. 34, 2357-2359.
Horne, D. W. (2003) J. Nutr. 133, 476-478.
Johnson, P. I., and Blusztajn, J. K. (1998) Neurochem. Res. 23, 583-587.
Jones, J. P., Meck, W., Williams, C. L., Wilson, W. A., and Swartzwelder, H. S. (1999) Brain Res. Dev. 118, 159-167.
Katzung, (2001) Basic & Clinical Pharmacology, 8th ed. Lange Medical Books/McGraw-Hill Medical Pub. Division, New York.
Kerins, D. M., Koury, M. J., Capdevila, A., Rana, S. & Wagner, C. (2001) Am. J. Clin. Nutr. 74, 723-729.
Kim, Y.-I., Miller, J. W., da Costa, K.-A., Nadeau, M., Smith, D., Selhub, J., Zeisel, S. H. & Mason, J. B. (1995) J. Nutr. 124, 2197-2203.
Koc, H., Mar, M. H., Ranasinghe, A., Swenberg, J. A., and Zeisel,
Kruskal, W., and Wallis, W. (1952) J. Am. Statist. Assoc. 47, 583-621.
Lowry, R. (2004) statistics. In Vasser University website /lowry/webtext accessed on Nov. 22, 2004.
Meck, W. H., Smith, R. A., and Williams, C. L. (1988) Dev. Psychobiol. 21, 339-353.
Meck, W., and Williams, C. (1997a) NeuroReport 8, 3053-3059.
Meck, W., and Williams, C. (1997b) NeuroReport 8, 2831-2835.
Meck, W., and Williams, C. (1997c) NeuroReport 8, 3045-3051.
Meck, W. H., and Williams, C. L. (1999) Brain Res. Dev. 118, 51-59.
Meck, W. H., and Williams, C. L. (2003) Neurosci. Biobehay. Rev. 27, 385-399.
Meyer, K., Fredriksen, A. & Ueland, P. M. (2004) Clin. Chem. 50, 391-402.
Millian, N. S. & Garrow, T. A. (1998) Arch. Biochem. Biophys. 356, 93-98.
Montoya, D. A., White, A. M., Williams, C. L., Blusztajn, J. K., Meck, W. H., and Swartzwelder, H. S. (2000) Brain Res. Dev. 123, 25-32.
Morin, I., Platt, R., Weisberg, I., Sabbaghian, N., Wu, Q., Garrow, T. A., and Rozen, R. (2003) Am. J. Med. Genet. A 119, 172-176.
PCT International Publication No. WO 93/25521.
Pomfret, E. A., da Costa, K.-A. & Zeisel, S. H. (1990) J. Nutr. Biochem. 1, 533-541.
Pyapali, G., Turner, D., Williams, C., Meck, W., and Swartzwelder, H. S. (1998) J. Neurophysiol. 79, 1790-1796.
Remington et al., (1975) Remington's Pharmaceutical Sciences, 15th ed. Mack Pub. Co., Easton, Pa.
Rozen, R. (1996) Clin. Invest. Med. 19, 171-178.
Saito, S., Iida, A., Sekine, A., Miura, Y., Sakamoto, T., Ogawa, C., Kawauchi, S., Higuchi, S., and Nakamura, Y. (2001) J. Hum. Genet. 46, 529-537.
Schwahn, B. C., Chen, Z., Laryea, M. D., Wendel, U., Lussier-Cacan, S., (2003) FASEB J. 17, 512-514.
Selhub, J., Seyoum, E., Pomfret, E. A. & Zeisel, S. H. (1991) Cancer Res. 51, 16-21.
Shaw, G. M., Schaffer, D., Velie, E. M., Morland, K. & Harris, J. A. (1995) Epidemiology 6, 219-226.
Shaw, G. M., Carmichael, S. L., Yang, W., Selvin, S., and Schaffer, D. M. (2004) Am. J. Epidemiol. 160, 102-109.
Shelnutt, K. P., Kauwell, G. P., Chapman, C. M., Gregory, J. F., 3rd, Maneval, D. R., Browdy, A. A., Theriaque, D. W., and Bailey, L. B. (2003) J. Nutr. 133, 4107-4111.
Shields, D. J., Agellon, L. B., and Vance, D. E. (2001) Biochim. Biophys. Acta 1532, 105-114.
Song, J., da Costa, K. A., Fischer, L., Kohlmeier, M., Kwock, L., Wang, S., and Zeisel, S. H. (2005) FASEB J. 19, 1266-1271.
Speight et al., (1997) Avery's Drug Treatment: A Guide to the Properties, Choice, Therapeutic Use and Economic Value of Drugs in Disease Management, 4th ed. Adis International, Auckland/Philadelphia.
Sunden, S., Renduchintala, M., Park, E., Miklasz, S. & Garrow, T. (1997) Arch. Biochem. Biophys. 345, 171-174.
Svardal, A. M., Ueland, P. M., Berge, R. K., Aarsland, A., Aarsaether, N., Lonning, P. E. & Refsum, H. (1988) Cancer. Chemother. Pharmacol. 21, 313-318.
Ting, A., and Morris, P. (1971) Vox Sang. 561-563.
Ubbink, J. B., Hayward Vermaak, W. J. & Bissbort, S. (1991) J. Chromatogr. 565, 441-446.
U.S. Pat. No. 5,326,902.
U.S. Pat. No. 5,234,933.
Vance, D. E., Walkey, C. J. & Cui, Z. (1997) Biochim. Biophys. Acta 1348, 142-150.
Varela-Moreiras, G., Selhub, J., da Costa, K.-A. & Zeisel, S. H. (1992) J. Nutr. Biochem. 3, 519-522.
Weisberg, I. S., Park, E., Ballman, K. V., Berger, P., Nunn, M., Suh, D. S., Breksa, A. P., 3rd, Garrow, T. A., and Rozen, R. (2003) Atherosclerosis 167, 205-214.
Wilcken, D., Wang, X., Sim, A. & McCredie, R. (1996) Arterioscler. Thromb. Vasc. Biol. 16, 878-882.
Williams, C. L., Meck, W. H., Heyer, D. D., and Loy, R. (1998) Brain Res. 794, 225-238.
Yang, E. K., Blusztajn, J. K., Pomfret, E. A. & Zeisel, S. H. (1988) Biochem. J. 256, 821-828.
Yao, Z. M. & Vance, D. E. (1988) J. Biol. Chem. 263, 2998-3004.
Yao, Z. M. & Vance, D. E. (1989) J. Biol. Chem. 264, 11373-11380.
Zeisel, S. H., Zola, T., da Costa, K.-A. & Pomfret, E. A. (1989) Biochem. J. 259, 725-729.
Zeisel, S. H., da Costa, K.-A., Franklin, P. D., Alexander, E. A., Lamont, J. T., Sheard, N. F. & Beiser, A. (1991) FASEB J. 5, 2093-2098.
Zeisel, S. H. & Blusztajn, J. K. (1994) Ann. Rev. Nutr. 14, 269-296.
Zeisel, S. H., Mar, M.-H., Zhou, Z.-W., and da Costa, K.-A. (1995) J. Nutr. 125, 3049-3054.
Zeisel, S. H., Mar, M. H., Howe, J. C. & Holden, J. M. (2003) J. Nutr. 133, 1302-1307.

It will be understood that various details of the subject matter disclosed herein can be changed without departing from the scope of the presently disclosed subject matter. Furthermore, the foregoing description is for the purpose of illustration only, and not for the purpose of limitation.

1-19. (canceled)

20. A method of treating one or more choline deficiency-associated health effects in a female human subject or a fetus carried by said subject, wherein the choline deficiency-associated health effects are associated with an insufficient dietary intake of choline, comprising:
(a) determining a genotype of the subject with respect to at least one choline metabolism gene;
(b) comparing the determined genotype of the subject with at least one reference genotype associated with increased susceptibility to developing one or more choline deficiency-associated health effects, wherein the reference genotype is at least one genotype of a choline metabolism gene;
(c) determining that the determined genotype of the subject is associated with an increased susceptibility of the subject to developing one or more choline deficiency-associated health effects, and
(d) administering to the subject an effective amount of a choline supplement composition.

21. (canceled)

22. The method of claim 20, wherein the choline metabolism gene is selected from the group consisting of phosphatidylethanolamine N-methyltransferase (PEMT), choline dehydrogenase (CHDH), 5,10-methylenetetrahydrofolate dehydrogenase 1 (MTHFD1), and combinations thereof.

23. The method of claim 22, wherein the reference genotype is selected from the group consisting of a PEMT genotype, a CHDH genotype, an MTHFD1 genotype, and combinations thereof.

24. The method of claim 23, wherein the reference genotype is a PEMT genotype.

25. The method of claim 24, wherein the determined genotype of the subject comprises at least one copy of a PEMT gene with a cytosine at the polymorphic position of rs12325817.

26. The method of claim 22 wherein the reference genotype is a CHDH genotype.

27. The method of claim 26, wherein the determined genotype of the subject comprises at least one copy of a CHDH gene with a thymine at the polymorphic position of rs12676.

28. (canceled)

29. (canceled)

30. The method of claim 23, wherein the reference genotype is a MTHFD1 genotype.

31. The method of claim 30, wherein the determined genotype of the subject comprises at least one copy of a MTHFD1 gene with an adenine at the polymorphic position of rs2236225.

32. The method of claim 20, wherein the one or more choline deficiency-associated health effects are selected from the group consisting of transmembrane signaling dysfunction, cholinergic neurotransmission dysfunction, lipid transport dysfunction, lipid metabolism dysfunction, organ dysfunction, liver dysfunction, fatty liver, congenital birth defects, and combinations thereof.

33. The method of claim 20, wherein the subject is a premenopausal female subject.

34. The method of claim 33, wherein the determined genotype of the subject comprises at least one copy of a PEMT gene with a cytosine at the polymorphic position of rs12325817, at least one copy of a MTHFD1 gene with an adenine at the polymorphic position of rs2236225, or combinations thereof.

35. The method of claim 33, wherein the subject is a pregnant subject and the one or more choline deficiency-associated health effects comprise one or more congenital birth defects to a fetus carried by the subject.

36. The method of claim 35, wherein the congenital birth defects comprise a neural tube defect.

37. The method of claim 20, wherein the subject is receiving substantially all nutritional sustenance parenterally.

38. The method of claim 37, wherein the one or more choline deficiency-associated health effects comprise liver dysfunction.

39-50. (canceled)


SEQUENCE LISTING (55 sequences)

SEQ ID NO: 1 - DNA, 51 bp, Homo sapiens
  misc_feature (26)..(26): s is g or c
cagcctggacaacatggtgacactcsgtctctactaaaaatacaaaaatag

SEQ ID NO: 2 - DNA, 51 bp, Homo sapiens
  misc_feature (26)..(26): y is c or t
cctgcagctcagcagacctcctggcygtggtgggtagctcctttcctttag

SEQ ID NO: 3 - DNA, 51 bp, Homo sapiens
  misc_feature (26)..(26): y is c or t
actcactcttcgtataggagagccaytatgtaggtgagggccaccagcacc

SEQ ID NO: 4 - DNA, 51 bp, Homo sapiens
  misc_feature (26)..(26): r is a or g
cgatcccctgcatgtggggctcaccragcagtgggatactcccgtctggac

SEQ ID NO: 5 - DNA, 51 bp, Homo sapiens
  misc_feature (26)..(26): y is c or t
ctatctctggccatactctcagccaycctaaaagtaaagccttgttgttag

SEQ ID NO: 6 - DNA, 51 bp, Homo sapiens
  misc_feature (26)..(26): r is a or g
cagtttccttgggtttcaagacccaraagcactcacttttatccttgtccc

SEQ ID NO: 7 - DNA, 51 bp, Homo sapiens
  misc_feature (26)..(26): r is a or g
agattaagcaactaacactgggggarcccagcagcagctgacatccacctg

SEQ ID NO: 8 - DNA, 51 bp, Homo sapiens
  misc_feature (26)..(26): r is a or g
tggggtgaagctcacaaccagcagargagtgagaagaggcagcccagcgtg

SEQ ID NO: 9 - DNA, 51 bp, Homo sapiens
  misc_feature (26)..(26): r is a or g
aggccctgccaccgagctgttcgttrcctcggctcccgggttcccagatct

SEQ ID NO: 10 - DNA, 51 bp, Homo sapiens
  misc_feature (26)..(26): k is g or t
gggaggtgccagatgtgccaagtgtkagggcagggcaaacaaggcaagacg

SEQ ID NO: 11 - DNA, 51 bp, Homo sapiens
  misc_feature (26)..(26): y is c or t
cagctaggtgataattactaatctcygctactgtgtatggaagacatcttg

SEQ ID NO: 12 - DNA, 51 bp, Homo sapiens
  misc_feature (26)..(26): y is c or t
ccagagagttctctgaaggagctaayaccagttagtgttttgaagagtagc

SEQ ID NO: 13 - DNA, 51 bp, Homo sapiens
  misc_feature (26)..(26): m is a or c
agcgcaggctctgagagccgggacgmgtacagctatgtggtggtgggcgcg

SEQ ID NO: 14 - DNA, 51 bp, Homo sapiens
  misc_feature (26)..(26): k is g or t
ctggaggccgggcccaaggacgtgckcgcggggagcaagcggctctcgtgg

SEQ ID NO: 15 - DNA, 51 bp, Homo sapiens
  misc_feature (26)..(26): y is c or t
tgggccaacaagcttgagtgcgatcyggtctgcaatgatggaggaattgcc

SEQ ID NO: 16 - DNA, 51 bp, Homo sapiens
  misc_feature (26)..(26): r is a or g
gccgcgtattgagttcagtggtgtcrttgatgtcaaaccatcctatttcct

SEQ ID NO: 17 - DNA, 51 bp, Homo sapiens
  misc_feature (26)..(26): s is g or c
agaaatttgacaccctggttggagasagaggggcccagctgagtggtgggc

SEQ ID NO: 18 - DNA, 51 bp, Homo sapiens
  misc_feature (26)..(26): r is a or g
ggcgagatcctcaccagaagactgcrgtcaatggcttttaaagcaatgcta

SEQ ID NO: 19 - DNA, 51 bp, Homo sapiens
  misc_feature (26)..(26): r is a or g
actaaatgatgaaaaggctgccactrgaatggccccaaatggctggaaatc

SEQ ID NO: 20 - DNA, 51 bp, Homo sapiens
  misc_feature (26)..(26): s is g or c
catagctcacggatcaggtctccccstcatgatgatagtatttggagagat

SEQ ID NO: 21 - DNA, 51 bp, Homo sapiens
  misc_feature (26)..(26): s is g or c
tcactgtttcttttctgtccagatastctcggcatttagtgacaaagaact

SEQ ID NO: 22 - DNA, 51 bp, Homo sapiens
  misc_feature (26)..(26): r is a or g
aggaggtcaaaaacagaggattgctrttgcccgagccctcatcagacaacc

SEQ ID NO: 23 - DNA, 51 bp, Homo sapiens
  misc_feature (26)..(26): y is c or t
agccactggacattgagtttctttgyttcttgaccatcgagaagctgaaaa

SEQ ID NO: 24 - DNA, 51 bp, Homo sapiens
  misc_feature (26)..(26): y is c or t
ttgaaggagaaggtgtctgcgggagycgatttcatcatcacgcagcttttc

SEQ ID NO: 25 - DNA, 51 bp, Homo sapiens
  misc_feature (26)..(26): m is a or c
tggggggaggagctgaccagtgaagmaagtgtctttgaagtctttgttctt

SEQ ID NO: 26 - DNA, 51 bp, Homo sapiens
  misc_feature (26)..(26): r is g or a
atgaaggagggcttggaggctgcccractgaaagctcacctgatgagccag

SEQ ID NO: 27 - DNA, 51 bp, Homo sapiens
  misc_feature (26)..(26): y is c or t
cccaaagtagcctaccaactcaccayctgcaaaatcgctccatacagccgc

SEQ ID NO: 28 - DNA, 51 bp, Homo sapiens
  misc_feature (26)..(26): w is a or t
aatcttggcttgtacaatggaccacwgtccccagaggaaatgagatgcaag

SEQ ID NO: 29 - DNA, 51 bp, Homo sapiens
  misc_feature (26)..(26): y is c or t
atttctgcagaaatatctggcaaacytaattcttcagtatctaatcgccgg

SEQ ID NO: 30 - DNA, 27 bp, Artificial Sequence
  synthesized oligonucleotide for forward primer for PCR amplification of PEMT
gagcacgtgagctgtcagtgccttttg

SEQ ID NO: 31 - DNA, 27 bp, Artificial Sequence
  synthesized oligonucleotide for reverse primer for PCR amplification of PEMT
ccaacctccttcatacaacagaggtcc

SEQ ID NO: 32 - DNA, 30 bp, Artificial Sequence
  synthesized oligonucleotide for forward primer for PCR amplification of PEMT
ggagttatggatctagggaactggagcagc

SEQ ID NO: 33 - DNA, 20 bp, Artificial Sequence
  synthesized oligonucleotide for forward primer for PCR amplification of PEMT
atttcaccctcctgaaagga

SEQ ID NO: 34 - DNA, 21 bp, Artificial Sequence
  synthesized oligonucleotide for reverse primer for PCR amplification of PEMT
tgaccaatctaagcccaggtt

SEQ ID NO: 35 - DNA, 21 bp, Artificial Sequence
  synthesized oligonucleotide for forward primer for PCR amplification of PEMT
tagattggtcatgggaggctt

SEQ ID NO: 36 - DNA, 20 bp, Artificial Sequence
  synthesized oligonucleotide for reverse primer for PCR amplification of PEMT
acaacatggtgacactccgt

SEQ ID NO: 37 - DNA, 20 bp, Artificial Sequence
  synthesized oligonucleotide for forward primer for PCR amplification of PEMT
tctcgaactcctgaccatca

SEQ ID NO: 38 - DNA, 19 bp, Artificial Sequence
  synthesized oligonucleotide for reverse primer for PCR amplification of PEMT
cccgtaatcccagcacttt

SEQ ID NO: 39 - DNA, 21 bp, Artificial Sequence
  synthesized oligonucleotide for forward primer for PCR amplification of PEMT
gaggaaaaagactctggcaca

SEQ ID NO: 40 - DNA, 21 bp, Artificial Sequence
  synthesized oligonucleotide for reverse primer for PCR amplification of PEMT
tttactccattgaggggtgca

SEQ ID NO: 41 - DNA, 18 bp, Artificial Sequence
  synthesized oligonucleotide for forward primer for PCR amplification of PEMT
tgatggatcccaggagga

SEQ ID NO: 42 - DNA, 21 bp, Artificial Sequence
  synthesized oligonucleotide for reverse primer for PCR amplification of PEMT
ggctttctgctacccagtaat

SEQ ID NO: 43 - DNA, 27 bp, Artificial Sequence
  synthesized oligonucleotide for reverse primer for PCR amplification of PEMT
acaacagaggtcccggctttctgctac

SEQ ID NO: 44 - DNA, 27 bp, Artificial Sequence
  synthesized oligonucleotide for forward primer for PCR amplification of PEMT
acaacagaggtcccggctttctgctac

SEQ ID NO: 45 - DNA, 23 bp, Artificial Sequence
  synthesized oligonucleotide for reverse primer for PCR amplification of PEMT
tcagagatcagcctggccaatat

SEQ ID NO: 46 - DNA, 20 bp, Artificial Sequence
  synthesized oligonucleotide for forward primer for PCR amplification of PEMT
atttttagtagagacggagt

SEQ ID NO: 47 - DNA, 20 bp, Artificial Sequence
  synthesized oligonucleotide for reverse primer for PCR amplification of PEMT
atcacaaggccaggagtcag

SEQ ID NO: 48 - DNA, 21 bp, Artificial Sequence
  synthesized oligonucleotide for forward primer for PCR amplification of PEMT
acttcctgggttgaagcgatt

SEQ ID NO: 49 - DNA, 19 bp, Artificial Sequence
  synthesized oligonucleotide for reverse primer for PCR amplification of PEMT
tttattctctggccgtgcc

SEQ ID NO: 50 - DNA, 21 bp, Artificial Sequence
  synthesized oligonucleotide for forward primer for PCR amplification of PEMT
ggagcactttgccccagaatc

SEQ ID NO: 51 - DNA, 20 bp, Artificial Sequence
  synthesized oligonucleotide for reverse primer for PCR amplification of PEMT
gacttggagccttcagagcg

SEQ ID NO: 52 - DNA, 29 bp, Artificial Sequence
  synthesized oligonucleotide for forward primer for PCR amplification of CHDH
agtcatctcattcccctccgtggatcaga

SEQ ID NO: 53 - DNA, 28 bp, Artificial Sequence
  synthesized oligonucleotide for reverse primer for PCR amplification of CHDH
tagcaccagttgtacctgtcgtcgcaca

SEQ ID NO: 54 - DNA, 23 bp, Artificial Sequence
  synthesized oligonucleotide for forward primer for PCR amplification of BHMT
gccactttgaccccaccattagt

SEQ ID NO: 55 - DNA, 22 bp, Artificial Sequence
  synthesized oligonucleotide for reverse primer for PCR amplification of BHMT
tgggaattctgggagatcgatg